(19) United States
(12) Patent Application Publication
(10) Pub. No.: US 2025/0262046 A1
(43) Pub. Date: Aug. 21, 2025

(54) OPHTHALMIC LENSES, OPHTHALMIC LENS COMPONENTS, AND APPARATUS, SYSTEMS, AND METHODS OF FORMING AN OPHTHALMIC LENS COMPONENT

(71) Applicant: Alcon Inc., Fribourg (CH)

(72) Inventors: Raza Ulabdin SHAH, San Francisco, CA (US); Alexander Jay DRURY, Boyceville, WI (US); Ge SONG, Foster City, CA (US)

(73) Assignee: Alcon Inc., Fribourg (CH)

(21) Appl. No.: 19/039,530

(22) Filed: Jan. 28, 2025

Related U.S. Application Data
(60) Provisional application No. 63/556,270, filed on Feb. 21, 2024.

(51) Int. Cl. A61F 2/16; A61F 2/50

(52) U.S. Cl. CPC A61F 2/1648; A61F 2/1643; A61F 2/5046; A61F 2002/169; A61F 2002/5053

(57) ABSTRACT

Disclosed are ophthalmic lenses, ophthalmic lens components, and methods, systems, and apparatus for manufacturing an ophthalmic lens component. In one aspect, disclosed is an intraocular lens, comprising an optic portion and a haptic coupled to the optic portion. The haptic can comprise a haptic anterior portion and a haptic posterior portion. The haptic anterior portion can have an anterior surface roughness and the haptic posterior portion can have a posterior surface roughness. The posterior surface roughness can be greater than the anterior surface roughness such that at least part of the haptic anterior portion is smoother than the haptic posterior portion.

### CROSS-REFERENCE TO RELATED APPLICATION

This application claims the benefit of U.S. Patent Application No. 63/556,270 filed on Feb. 21, 2024, the content of which is incorporated herein by reference in its entirety.

### TECHNICAL FIELD

The present disclosure relates generally to the field of ophthalmic devices, and, more specifically, to ophthalmic lenses, ophthalmic lens components, and apparatus, systems, and methods of forming an ophthalmic lens component.

### BACKGROUND

A cataract is a condition involving the clouding over of the normally clear lens of a patient's eye. Cataracts occur as a result of aging, hereditary factors, trauma, inflammation, metabolic disorders, or exposure to radiation. Age-related cataract is the most common type of cataracts. In treating a cataract, the surgeon removes the native crystalline lens matrix from the patient's capsular bag and replaces it with an intraocular lens (IOL).

Traditional IOLs provide one or more selected focal lengths that allow the patient to have distance vision. However, after cataract surgery, patients with traditional IOLs often require glasses or other corrective eyewear for certain activities since the eye can no longer undertake accommodation (or change its optical power) to maintain a clear image of an object or focus on an object as its distance varies. Newer IOLs such as accommodating IOLs (AIOLs), allow the eye to regain at least some focusing ability. AIOLs use forces available in the eye to change some portion of the optical system in order to refocus the eye on distant or near targets.

There may be a need to adjust IOLs post-operatively or after implantation within the eye of a subject. In some instances, an implanted IOL may be adjusted using laser treatments. When laser treatments are used as part of the post-operative adjustment procedure, patient safety becomes paramount.

Therefore, solutions are needed which allow a medical professional to safely and accurately adjust an IOL post-operatively. Such a solution should be designed with clinical considerations in mind.

### SUMMARY

Disclosed herein are ophthalmic lenses, ophthalmic lens components, and apparatus, systems, and methods of forming an ophthalmic lens component. In some embodiments, disclosed is an intraocular lens comprising an optic portion and a haptic coupled to the optic portion. The haptic can comprise a haptic anterior portion and a haptic posterior portion. The haptic anterior portion can have an anterior surface roughness and the haptic posterior portion can have a posterior surface roughness. The posterior surface roughness can be greater than the anterior surface roughness such that at least part of the haptic anterior portion is smoother than the haptic posterior portion.

In some embodiments, the anterior surface roughness can have a roughness average (Ra) of between about 0.10 μm and about 0.35 μm.

In some embodiments, the anterior surface roughness can be graded at a Society of the Plastics Industry (SPI) grade of between B1 and C1.

In some embodiments, the posterior surface roughness can have a roughness average (Ra) of between about 0.25 μm and about 0.60 μm.

In some embodiments, the posterior surface roughness can be graded at a Society of the Plastics Industry (SPI) grade of between B3 and C3.

In some embodiments, a thickness or depth of the haptic anterior portion can be between about 600 μm and about 850 μm.

In some embodiments, a thickness or depth of the haptic posterior portion can be between about 600 μm and about 850 μm.

In some embodiments, the optic portion can comprise an optic fluid chamber. The haptic can comprise a haptic lumen extending through at least part of the haptic and in fluid communication with the optic fluid chamber.

In some embodiments, at least part of the haptic can be made of a composite material.

In some embodiments, the composite material can be positioned anterior to the haptic posterior portion.

In some embodiments, the composite material can be positioned posterior to the haptic anterior portion.

In some embodiments, the composite material can be configured to change shape in response to receiving laser light directed at the composite material. A base power of the optic portion can be configured to change in response to the laser light directed at the composite material.

In some embodiments, the haptic posterior portion can be configured to diffuse the laser light directed at the haptic.

In some embodiments, at least part of the haptic anterior portion can be characterized by a smooth texture or finish.

In some embodiments, the smooth texture or finish can be imparted by a first molding surface of a first mold component.

In some embodiments, at least part of the haptic posterior portion can be characterized by a frosted texture or finish.

In some embodiments, the frosted texture or finish can be imparted by a second molding surface of a second mold component configured to couple to or mate with the first mold component.

In some embodiments, the haptic anterior portion can be substantially transparent.

In some embodiments, the haptic posterior portion can be substantially translucent.

Also disclosed is a system for manufacturing a lens component. The system can comprise a first mold component comprising a first molding surface defined by a first partial component cavity and a second mold component comprising a second molding surface defined by a second partial component cavity. At least part of the first molding surface can have a first surface roughness and at least part of the second molding surface can have a second surface roughness. The second surface roughness can be greater than the first surface roughness such that at least part of the first molding surface can be smoother than the second molding surface. The first mold component can be configured to be mated to the second mold component to form an assembled mold. A mold cavity within the assembled mold can be configured to receive a lens component material for curing into a molded lens component.

In some embodiments, the first surface roughness can have a roughness average (Ra) of between about 0.10 μm and about 0.35 μm.

In some embodiments, the first surface roughness can be graded at a Society of the Plastics Industry (SPI) grade of between B1 and C1.

In some embodiments, the second surface roughness can have a roughness average (Ra) of between about 0.25 μm and about 0.60 μm.

In some embodiments, the second surface roughness can be graded at a Society of the Plastics Industry (SPI) grade of between B3 and C3.

In some embodiments, the system can further comprise a soluble core component configured to be placed on either the first molding surface or the second molding surface. A segment of the soluble core component can be configured to be disposed within the mold cavity formed by the first partial component cavity and the second partial component cavity when the first mold component is mated to the second mold component. At least part of the soluble core component can be configured to be dissolved in a solvent when the molded lens component is immersed in the solvent.

In some embodiments, the soluble core component can be made in part of a dissolvable material.

In some embodiments, the lens component material can be configured to be cured within the assembled mold into the molded lens component. The molded lens component can be configured to be removed from either the first mold component or the second mold component when the first mold component is separated from the second mold component after curing. At least a segment of the soluble core component can be within the molded lens component when the molded lens component is removed from either the first mold component or the second mold component.

In some embodiments, the lens component can be a haptic of a lens and a haptic chamber or haptic lumen can be formed within the haptic when the soluble core component is dissolved.

In some embodiments, at least one of the first mold component and the second mold component can be made of a polymeric material. The polymeric material can be at least one of polymethyl pentene and polypropylene.

Further disclosed is a method of manufacturing a lens component. The method can comprise providing a first mold component comprising a first molding surface defined by a first partial component cavity and providing a second mold component comprising a second molding surface defined by a second partial component cavity. At least part of the first molding surface can have a first surface roughness. At least part of the second molding surface can have a second surface roughness. The second surface roughness can be greater than the first surface roughness such that the first molding surface is smoother than the second molding surface. The method can also comprise mating a second mold component to the first mold component to form an assembled mold, introducing a lens component material into a mold cavity, and curing the lens component material within the assembled mold to form a molded lens component.

In some embodiments, the first surface roughness can have a roughness average (Ra) of between about 0.10 μm and about 0.35 μm.

In some embodiments, the first surface roughness can be graded at a Society of the Plastics Industry (SPI) grade of between B1 and C1.

In some embodiments, the second surface roughness can have a roughness average (Ra) of between about 0.25 μm and about 0.60 μm.

In some embodiments, the second surface roughness can be graded at a Society of the Plastics Industry (SPI) grade of between B3 and C3.

In some embodiments, the method can further comprise placing at least part of a soluble core component on either the first molding surface or the second molding surface. A segment of the soluble core component can be disposed within the mold cavity formed by the first partial component cavity and the second partial component cavity. The method can also comprise immersing the molded lens component in a solvent to dissolve the soluble core component.

In some embodiments, the method can further comprise separating the first mold component from the second mold component after curing the lens component material to form the molded lens component and removing the molded lens component from the first mold component or the second mold component. At least a segment of the soluble core component can be within the molded lens component when the molded lens component is removed.

In some embodiments, the lens component can be a haptic of a lens.

In some embodiments, a haptic chamber or haptic lumen can be formed within the haptic when the soluble core component is dissolved.

In some embodiments, the lens component material can be configured to be cured within the assembled mold into the molded lens component. The molded lens component can be configured to be removed from either the first mold component or the second mold component when the first mold component is separated from the second mold component after curing. At least a segment of the soluble core component can be within the molded lens component when the molded lens component is removed from either the first mold component or the second mold component.

Also disclosed is an intraocular lens haptic comprising a haptic anterior portion and a haptic posterior portion. The haptic anterior portion can have an anterior surface roughness and the haptic posterior portion can have a posterior surface roughness. The posterior surface roughness can be greater than the anterior surface roughness such that at least part of the haptic anterior portion is smoother than the haptic posterior portion.

In some embodiments, the anterior surface roughness can have a roughness average (Ra) of between about 0.10 μm and about 0.35 μm.

In some embodiments, the anterior surface roughness can be graded at a Society of the Plastics Industry (SPI) grade of between B1 and C1.

In some embodiments, the posterior surface roughness can have a roughness average (Ra) of between about 0.25 μm and about 0.60 μm.

In some embodiments, the posterior surface roughness can be graded at a Society of the Plastics Industry (SPI) grade of between B3 and C3.

In some embodiments, a thickness or depth of the haptic anterior portion can be between about 600 μm and about 830 μm.

In some embodiments, a thickness or depth of the haptic posterior portion can be between about 600 μm and about 830 μm.

In some embodiments, at least part of the intraocular lens haptic can be made of a composite material.

In some embodiments, the composite material can be positioned anterior to the haptic posterior portion.

In some embodiments, the composite material can be positioned posterior to the haptic anterior portion.

In some embodiments, the composite material can be configured to change shape in response to receiving laser light directed at the composite material.

In some embodiments, the haptic posterior portion can be configured to diffuse the laser light directed at the intraocular lens haptic.

In some embodiments, at least part of the haptic anterior portion can be characterized by a smooth texture or finish.

In some embodiments, the smooth texture or finish can be imparted by a first molding surface of a first mold component.

In some embodiments, at least part of the haptic posterior portion can be characterized by a frosted texture or finish.

In some embodiments, the frosted texture or finish can be imparted by a second molding surface of a second mold component configured to couple to or mate with the first mold component.

In some embodiments, the haptic anterior portion can be substantially transparent.

In some embodiments, the haptic posterior portion can be substantially translucent.

### BRIEF DESCRIPTION OF THE DRAWINGS

FIG. 1A illustrates a top plan view of an embodiment of an intraocular lens.

FIGS. 1B and 1C illustrate sectional views of the intraocular lens.

FIG. 2A illustrates an embodiment of a first mold component that can be used to make part of the intraocular lens.

FIG. 2B illustrates an embodiment of a second mold component that can be used to make part of the intraocular lens.

FIG. 2C illustrates an embodiment of a clamping member for securing together the first mold component to the second mold component.

FIG. 2D illustrates an embodiment of a soluble core component placed on the first mold component.

FIG. 2E illustrates an assembled mold comprising the first mold component mated to the second mold component.

FIG. 3A illustrates a perspective view of an embodiment of a soluble core component.

FIG. 3B illustrates a perspective view of another embodiment of a soluble core component.

FIG. 4 illustrates an embodiment of a system for manufacturing part of the intraocular lens.

FIG. 5A is an image showing a top-down view of an anterior portion of one embodiment of a haptic.

FIG. 5B is an image showing a top-down view of a posterior portion of the haptic shown in FIG. 5A.

FIG. 6 is an image showing a close-up side view of part of the haptic of FIG. 5A.

FIG. 7A is an image showing part of the first mold component.

FIG. 7B is an image showing a close-up view of part of the first mold component.

FIG. 8A is an image showing part of the second mold component.

FIG. 8B is an image showing a close-up view of part of the second mold component.

### DETAILED DESCRIPTION

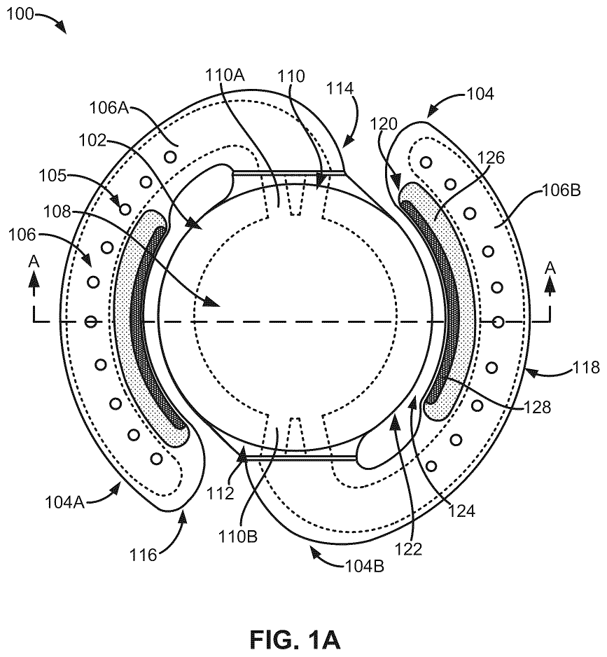

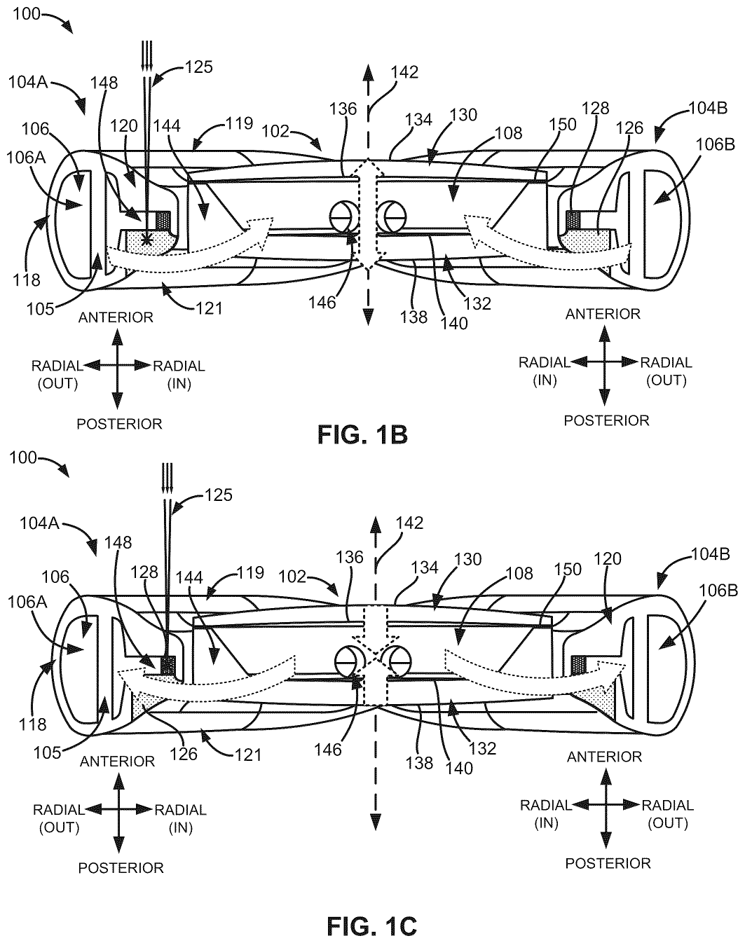

Disclosed herein are intraocular lenses (IOLs) and apparatus, systems, and methods of forming components of IOLs. For example, FIGS. 1A-1C illustrate an embodiment of an IOL 100 that can be made using lens components (e.g., haptics) formed using the apparatus, systems, and methods described herein. In some embodiments, the IOL 100 can be an adjustable IOL such as an accommodating IOL (AIOL). The IOL 100 can be implanted within a subject to correct for defocus aberration, corneal astigmatism, spherical aberration, or a combination thereof.

FIG. 1A illustrates a top plan view of the IOL 100. The IOL 100 can comprise an optic portion 102 and one or more haptics 104 comprising at least a first haptic 104A and a second haptic 104B coupled to and extending peripherally from the optic portion 102. The IOL 100 can be positioned within a native capsular bag in which a native lens has been removed.

When implanted within the native capsular bag, the optic portion 102 can be adapted to refract light that enters the eye onto the retina. The one or more haptics 104 can be configured to engage the capsular bag and be adapted to deform in response to ciliary muscle movement (e.g., muscle relaxation, muscle contraction, or a combination thereof) in connection with capsular bag reshaping.

Each of the haptics 104 can comprise a haptic lumen 106 extending through at least part of the haptic 104. For example, the first haptic 104A can comprise a first haptic lumen 106A extending through at least part of the first haptic 104A and the second haptic 104B can comprise a second haptic lumen 106B extending through at least part of the second haptic 104B. The haptic lumen 106 (e.g., any of the first haptic lumen 106A or the second haptic lumen 106B) can be in fluid communication with or fluidly connected to an optic fluid chamber 108 within the optic portion 102.

The optic fluid chamber 108 can be in fluid communication with the one or more haptic lumens 106 through one or more fluid channels 110. The fluid channels 110 can be conduits or passageways fluidly connecting the optic fluid chamber 108 to the haptic lumens 106. The fluid channels 110 can be spaced apart from one another. For example, a pair of fluid channels 110 can be spaced apart between about 0.1 mm to about 1.0 mm. In some embodiments, each of the fluid channels 110 can have a diameter of between about 0.4 mm to about 0.6 mm.

The haptics 104 can be coupled to the optic portion 102 at a reinforced portion 112. The reinforced portion 112 can serve as a haptic-optic interface. The pair of fluid channels 110 can be defined or formed within part of the reinforced portion 112.

As shown in FIG. 1A, the optic fluid chamber 108 can be in fluid communication with the first haptic lumen 106A through a first pair of fluid channels 110A. The optic fluid chamber 108 can also be in fluid communication with the second haptic lumen 106B through a second pair of fluid channels 110B.

In some embodiments, the first pair of fluid channels 110A and the second pair of fluid channels 110B can be positioned substantially on opposite sides of the optic portion 102. The first pair of fluid channels 110A can be positioned substantially diametrically opposed to the second pair of fluid channels 110B. The first pair of fluid channels 110A and the second pair of fluid channels 110B can be defined or can extend through part of the optic portion 102. The first pair of fluid channels 110A and the second pair of fluid channels 110B can be defined or can extend through a posterior element 132 of the optic portion 102 (see, e.g., FIGS. 1B and 1C).

FIG. 1A also illustrates that each of the haptics 104 (e.g., any of the first haptic 104A or the second haptic 104B) can have a proximal attachment end 114 and a distal free end 116. A haptic fluid port can be defined at the proximal attachment end 114 of the haptic 104. The haptic fluid port can serve as an opening of the haptic lumen 106. Fluid within the haptic lumen 106 can flow out of the haptic lumen 106 through the haptic fluid port and into the optic fluid chamber 108 via the fluid channels 110 when the haptic 104 is coupled to the optic portion 102. Similarly, fluid within the optic fluid chamber 108 can flow out of the optic fluid chamber 108 through the pair of fluid channels 110 and into the haptic lumen 106 through the haptic fluid port.

Each of the haptics 104 can comprise a radially-outer haptic wall 118 and a radially-inner haptic wall 120. The radially-outer haptic wall 118 can be configured to face and contact an inner surface of a patient's capsular bag when the IOL 100 is implanted within the capsular bag. The radially-inner haptic wall 120 can be configured to face an outer peripheral surface 122 of the optic portion 102.

The IOL 100 can be implanted or introduced into a patient's capsular bag after a native lens has been removed from the capsular bag. The patient's capsular bag is connected to zonule fibers which are connected to the patient's ciliary muscles. The capsular bag is elastic and ciliary muscle movements can reshape the capsular bag via the zonule fibers. For example, when the ciliary muscles relax, the zonules are stretched. This stretching pulls the capsular bag in the generally radially outward direction due to radially outward forces. This pulling of the capsular bag causes the capsular bag to elongate, creating room within the capsular bag. When the patient's native lens is present in the capsular bag, the native lens normally becomes flatter (in the anterior-to-posterior direction), which reduces the power of the lens, allowing for distance vision. In this configuration, the patient's native lens is said to be in a disaccommodated state or undergoing disaccommodation.

When the ciliary muscles contract, however, as occurs when the eye is attempting to focus on near objects, the radially inner portion of the muscles move radially inward, causing the zonules to slacken. The slack in the zonules allows the elastic capsular bag to contract and exert radially inward forces on a lens within the capsular bag. When the patient's native lens is present in the capsular bag, the native lens normally becomes more curved (e.g., the anterior part of the lens becomes more curved), which gives the lens more power, allowing the eye to focus on near objects. In this configuration, the patient's native lens is said to be in an accommodated state or undergoing accommodation.

When the IOL 100 is implanted into a patient's capsular bag, the radially-outer haptic wall 118 of the haptics 104 can directly engage with or be in physical contact with the portion of the capsular bag that is connected to the zonules or zonule fibers. Therefore, the radially-outer haptic wall 118 can be configured to respond to capsular bag reshaping forces that are applied radially when the zonules relax and stretch as a result of ciliary muscle movements.

When the ciliary muscles contract, the peripheral region of the elastic capsular bag reshapes and applies radially inward forces on the radially-outer haptic wall 118 of each of the haptics 104. The radially-outer haptic wall 118 can then deform or otherwise changes shape and this deformation or shape-change can cause the volume of the haptic lumen 106 to decrease. When the volume of the haptic lumen 106 decreases, the fluid within the haptic lumen 106 is moved or pushed into the optic fluid chamber 108.

The optic portion 102 can change shape in response to fluid entering the optic fluid chamber 108 from the haptic lumen 106. This can increase the base power or base spherical power of the IOL 100 and allow a patient with the IOL 100 implanted within the eye of the patient to focus on near objects. In this state, the IOL 100 can be considered to have undergone accommodation.

When the ciliary muscles relax, the peripheral region of the elastic capsular bag is stretched radially outward and the capsular bag elongates. The radially-outer haptic wall 118 of the haptics 104 can be configured to respond to this capsular bag reshaping by returning to its non-deformed or non-stressed configuration. This causes the volume of the haptic lumen 106 to increase or return to its non-deformed volume. This increase in the volume of the haptic lumen 106 can cause the fluid within the optic fluid chamber 108 to be drawn out or otherwise flow out of the optic fluid chamber 108 and back into the haptic lumen 106. As discussed previously, fluid moves out of the optic fluid chamber 108 into the haptic lumen 106 through the same fluid channels 110 formed within the optic portion 102.

As previously discussed, the optic portion 102 can change shape in response to fluid exiting the optic fluid chamber 108 and into the haptic lumen 106. This can decrease the base power or base spherical power of the IOL 100 and allow a patient with the IOL 100 implanted within the eye of the patient to focus on distant objects or provide for distance vision. In this state, the IOL 100 can be considered to have undergone disaccommodation.

In some embodiments, the IOL 100 can be designed such that a gap 124 or void space radially separates the radially-inner haptic wall 120 of the haptic 104 from the outer peripheral surface 122 of the optic portion 102. This can allow portions of the haptic 104 to change shape or expand in response to an external energy (e.g., laser energy) directed at the haptic 104.

FIG. 1A also illustrates that one or more portions of each of the haptics 104 can be made of a composite material. The composite material can comprise or be made in part of an energy absorbing constituent, a plurality of expandable components, and a cross-linked copolymer used to make the rest of the haptic 104. The portions of the haptics 104 made of the composite material can be configured to change shape (e.g., expand) in response to the laser light 125 (see, e.g., FIGS. 1B-1C) directed at the composite material. Depending on where the composite material is positioned or integrated within each of the haptics 104, the composite material can act as a lumen filler 126 to take up space within the haptic lumen 106 and/or a lumen expander 128 to create more space within the haptic lumen 106.

When laser light 125 is applied to the composite material configured as the lumen filler 126, the composite material can expand and the expansion of the composite material in this instance can decrease a volume of the haptic lumen 106 and cause fluid within the haptic lumen 106 to be displaced into the optic fluid chamber 108. This can cause the optic portion to change shape (e.g., cause the anterior or posterior elements of the optic portion 102 to become more curved) leading to an increase in the base power of the optic portion 102.

Alternatively, when the laser light 125 is applied to the composite material configured as the lumen expander 128, the composite material can expand and the expansion of the composite material in this instance can increase a volume of the haptic lumen 106 and cause fluid within the optic fluid chamber 108 to be drawn into the haptic lumen 106. This can also cause the optic portion 102 to change shape (e.g., cause the anterior or posterior elements of the optic portion 102 to become less curved or flatter) leading to a decrease in the base power of the optic portion 102.

Each of the haptics 104 can comprise a plurality of haptic isolators 105 that can counteract or reduce the effects of unintended shape changes or deformations caused by the application of laser light 125 to the haptics 104.

Although IOLS (including AIOLs) are depicted and described in this disclosure, any reference to an IOL or AIOL can also refer to one of the IOLs or AIOLs discussed and depicted in the following U.S. publications: U.S. Pat. Pub. No. 2021/0100652; U.S. Pat. Pub. No. 2021/0100650; U.S. Pat. Pub. No. 2020/0337833; and U.S. Pat. Pub. No. 2018/0153682; and in the following issued U.S. patents: U.S. Pat. Nos. 11,426,270; 10,433,949; 10,299,913; 10,195,020; and 8,968,396, the contents of which are incorporated herein by reference in their entireties.

FIGS. 1B and 1C illustrate cross-sectional views of the IOL 100 of FIG. 1A taken along cross-section A-A. As shown in FIGS. 1B and 1C, the optic portion 102 can comprise an anterior element 130 and a posterior element 132. The fluid-filled optic fluid chamber 108 can be defined in between the anterior element 130 and the posterior element 132.

The anterior element 130 can comprise an anterior optical surface 134 and an anterior inner surface 136 opposite the anterior optical surface 134. The posterior element 132 can comprise a posterior optical surface 138 and a posterior inner surface 140 opposite the posterior optical surface 138. Any of the anterior optical surface 134, the posterior optical surface 138, or a combination thereof can be considered and referred to as an external optical surface. The anterior inner surface 136 and the posterior inner surface 140 can face the optic fluid chamber 108. At least part of the anterior inner surface 136 and at least part of the posterior inner surface 140 can serve as chamber walls of the optic fluid chamber 108.

As shown in FIGS. 1B and 1C, the optic portion 102 can have a lens optical axis 142 extending in an anterior-to-posterior direction through a center of the optic portion 102. The lens optical axis 142 can extend through the centers of both the anterior element 130 and the posterior element 132.

The thickness of the anterior element 130 can be greater at or near the lens optical axis than at the periphery of the anterior element 130. In some embodiments, the thickness of the anterior element 130 can increase gradually from the periphery of the anterior element 130 toward the lens optical axis 142.

In certain embodiments, the thickness of the anterior element 130 at or near the lens optical axis 142 can be between about 0.45 mm and about 0.55 mm. In these and other embodiments, the thickness of the anterior element 130 near the periphery can be between about 0.20 mm and about 0.40 mm. Moreover, the anterior inner surface 136 of the anterior element 130 can have less curvature or be flatter than the anterior optical surface 134.

The thickness of the posterior element 132 can be greater at or near the lens optical axis 142 than portions of the posterior element 132 radially outward from the lens optical axis 142 but prior to reaching a raised periphery 144 of the posterior element 132. The thickness of the posterior element 132 can gradually decrease from the lens optical axis 142 to portions radially outward from the lens optical axis 142 (but prior to reaching the raised periphery 144). As shown in FIGS. 1B and 1C, the thickness of the posterior element 132 can increase once again from a radially inner portion of the raised periphery 144 to a radially outer portion of the raised periphery 144.

In certain embodiments, the thickness of the posterior element 132 at or near the lens optical axis 142 can be between about 0.45 mm and about 0.55 mm. In these and other embodiments, the thickness of the posterior element 132 radially outward from the lens optical axis 142 (but prior to reaching the raised periphery 144) can be between about 0.20 mm and about 0.40 mm. The thickness of the posterior element 132 near the radially outer portion of the raised periphery 144 can be between about 1.00 mm and 1.15 mm. Moreover, the posterior inner surface 140 of the posterior element 132 can have less curvature or be flatter than the posterior optical surface 138.

The optic portion 102 can have a base power or base spherical power. The base power of the optic portion 102 can be configured to change based on an internal fluid pressure within the fluid-filled optic fluid chamber 108. The base power of the optic portion 102 can be configured to increase or decrease as fluid enters or exits the fluid-filled optic fluid chamber 108.

The base power of the optic portion 102 can be configured to increase as fluid enters the fluid-filled optic fluid chamber 108 from the haptic lumen(s) 106, as depicted in FIG. 1B using the curved broken-line arrows. For example, the anterior element 130 of the optic portion 102 can be configured to increase its curvature in response to the fluid entering the optic fluid chamber 108. Also, for example, the posterior element 132 of the optic portion 102 can be configured to increase its curvature in response to the fluid entering the optic fluid chamber 108. In further embodiments, both the anterior element 130 and the posterior element 132 can be configured to increase their curvatures in response to the fluid entering the optic fluid chamber 108.

The base power of the optic portion 102 can be configured to decrease as fluid exits or is drawn out of the fluid-filled optic fluid chamber 108 into the haptic lumen(s) 106, as depicted in FIG. 1C using the curved broken-line arrows. For example, the anterior element 130 of the optic portion 102 can be configured to decrease its curvature (or flatten out) in response to the fluid exiting the optic fluid chamber 108. Also, for example, the posterior element 132 of the optic portion 102 can be configured to decrease its curvature (or flatten out) in response to the fluid exiting the optic fluid chamber 108. In further embodiments, both the anterior element 130 and the posterior element 132 can be configured to decrease their curvatures in response to the fluid exiting the optic fluid chamber 108.

It should be noted that although FIGS. 1B and 1C illustrate fluid entering and exiting the optic fluid chamber 108 from the haptic lumens 106 using the curved broken-line arrows, fluid enters and exits the optic fluid chamber 108 via the fluid channels 110 and apertures 146 defined along the posterior element 132. The apertures 146 can be holes or openings defined along the posterior element 132 that serve as terminal ends of the fluid channels 110. When the IOL 100 comprises a pair of fluid channels 110, the pair of apertures 146 serving as ends of the fluid channels 110 can be spaced apart from one another between about 0.1 mm to about 1.0 mm.

As shown in FIGS. 1B and 1C, one or more portions of the IOL 100 can be made of a composite material designed to respond to an external energy, such as laser light 125, applied to the composite material. For example, one or more portions of each of the haptics 104 of the IOL 100 can be made of the composite material.

In some embodiments, the laser light 125 can be a green laser light with a wavelength between about 480 nm and 650 nm (e.g., 532 nm). In these embodiments, the laser generating the laser light 125 can be a neodymium-doped yttrium aluminum garnet (Nd:YAG).

In other embodiments, the laser light 125 can have a wavelength between 1030 nm and 1035 nm. In these embodiments, the laser generating the laser light 125 can be a femtosecond laser.

Depending on where the composite material is positioned or integrated within each of the haptics 104 and the composition of the composite material, the composite material can act as a lumen filler 126 or a lumen expander 128.

For example, the lumen filler 126 can be a portion of the haptic 104 made of the composite material that is designed to decrease a volume of the haptic lumen 106 in response to an external energy (e.g., laser light 125) directed at the lumen filler 126. The lumen expander 128 can be a portion of the haptic 104 made of the composite material that is designed to increase a volume of the haptic lumen 106 in response to an external energy (e.g., laser light 125) directed at the lumen expander 128.

As shown in FIGS. 1B and 1C, each of the haptics 104 can comprise a channel 148. The channel 148 can be defined within part of the radially-inner haptic wall 120. For example, the channel 148 can extend partially into the radially-inner haptic wall 120. The channel 148 can be in fluid communication with the haptic lumen 106 or be considered part of the haptic lumen 106.

In some embodiments, the lumen filler 126 can be positioned posterior to the channel 148. In these embodiments, the lumen filler 126 can replace or act as the posterior portion of the radially-inner haptic wall 120. The lumen filler 126 can also be positioned radially inward of the portion of the haptic lumen 106 that is not the channel 148.

At least part of the lumen filler 126 can be in fluid communication with the channel 148. For example, at least part of an anterior portion or layer of the lumen filler 126 can be in fluid communication with or otherwise exposed to the channel 148.

It should be understood by one of ordinary skill in the art that even though different colored shading is used to differentiate the lumen filler 126 from the lumen expander 128 in the figures (that is, a darker shading pattern is used to depict the lumen expander 128 and a lighter shading pattern is used to depict the lumen filler 126), both the lumen filler 126 and the lumen expander 128 can be made of the same composite material or refer to different parts/features of the same block of composite material.

In other embodiments, the lumen filler 126 and the lumen expander 128 can be made of different types of composite materials. In these embodiments, the lumen filler 126 can be made of a first type of composite material and the lumen expander 128 can be made of a second type of composite material. In certain embodiments, the lumen filler 126 and the lumen expander 128 can be made of different colored composite materials. For example, the composite material can comprise an energy absorbing constituent such as an energy absorbing pigment or dye.

As a more specific example, either the lumen filler 126 or the lumen expander 128 can be made of a composite material comprising a black-colored energy absorbing pigment such as graphitized carbon black. In this example, if one of the lumen filler 126 or the lumen expander 128 is made of a composite material comprising graphitized carbon black, the other can be made of another type of composite material comprising a red-colored energy absorbing pigment such as an azo dye (e.g., Disperse Red 1 dye).

As shown in FIG. 1B, an external energy such as laser light 125 can be directed at the lumen filler 126 to cause at least part of the lumen filler 126 to expand and grow in size. For example, this expansion can manifest itself as a protuberance growing or jutting out of the lumen filler 126. For example, when laser light 125 is directed at the anterior portion or layer of the lumen filler 126 in fluid communication with or otherwise exposed to the channel 148, a protuberance can grow out of the anterior portion and into the channel 148. Since the channel 148 is in fluid communication with the haptic lumen 106 (or is considered part of the haptic lumen 106), the volume of the haptic lumen 106 can decrease in response to the formation of the protuberance. This can cause fluid within the haptic lumen 106 to be pushed or otherwise displaced into the optic fluid chamber 108. As a result, at least one of the anterior element 130 and the posterior element 132 can increase its curvature and the base power of the optic portion 102 can increase in response to the laser stimulus directed at the lumen filler 126.

An external energy such as the laser light 125 (e.g., laser pulses) can be directed at the lumen expander 128 to cause at least part of the lumen expander 128 to expand and grow in size. This expansion can manifest itself as an expansion of the channel 148. For example, when laser light 125 is directed at the lumen expander 128, the lumen expander 128 can grow in size and enlarge the channel 148. Since the channel 148 is in fluid communication with the haptic lumen 106 (or is considered part of the haptic lumen 106), the volume of the haptic lumen 106 can increase in response to the growth of the lumen expander 128. This can cause fluid to be drawn out of the optic fluid chamber 108 and into the haptic lumen 106. As a result, at least one of the anterior element 130 and the posterior element 132 can decrease its curvature and the base power of the optic portion 102 can decrease in response to the laser light 125 (e.g., laser pulses) directed at the lumen expander 128.

Each of the haptics 104 can comprise a plurality of haptic isolators 105 that can counteract or reduce the effects of unintended shape changes or deformations caused by the application of laser light 125 to the haptics 104.

Each of the haptics 104 can comprise a haptic anterior portion 119 and a haptic posterior portion 121. The haptic anterior portion 119 can comprise an exterior surface of the haptic 104 along an anterior side of the haptic 104 and at least part of an anterior wall of the haptic 104. The haptic posterior portion 121 can comprise an exterior surface of the haptic 104 along a posterior side of the haptic 104 and at least part of a posterior wall of the haptic 104.

The haptic anterior portion 119 can have an anterior portion thickness or depth. In some embodiments, the anterior portion thickness or depth can be between about 600 μm and about 850 μm (e.g., about 830 μm).

The haptic posterior portion 121 can have a posterior portion thickness or depth. In some embodiments, the posterior portion thickness or depth can be between about 600 μm and about 850 μm (e.g., about 830 μm).

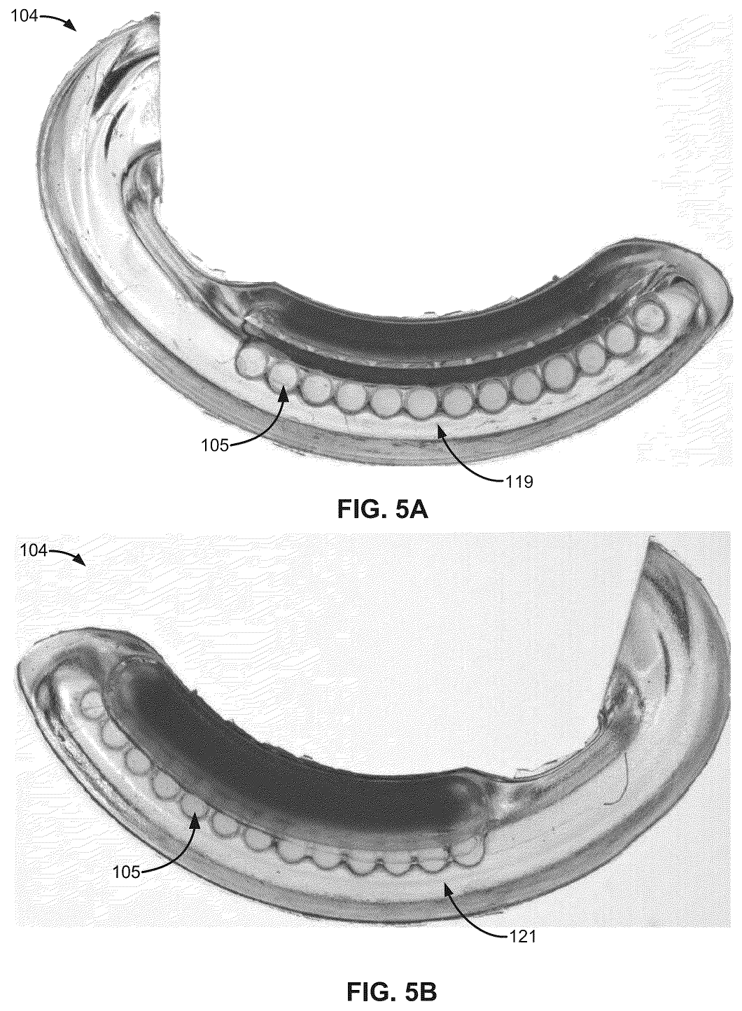

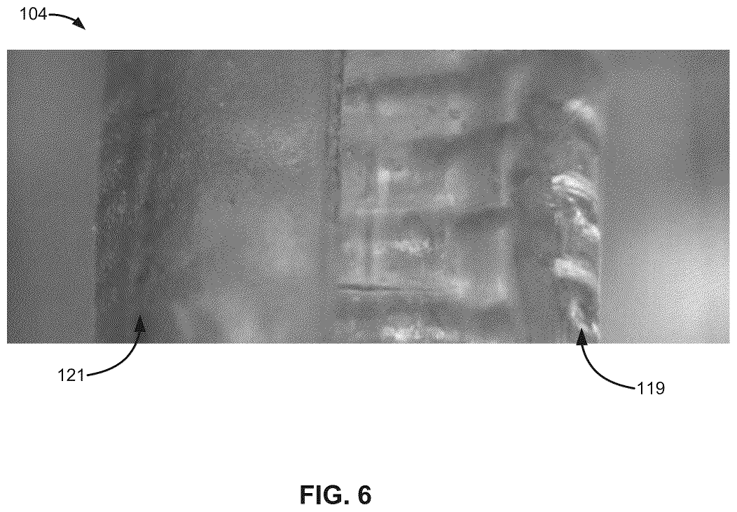

As will be discussed in more detail in relation to FIGS. 5A-5B and FIG. 6, the haptic 104 can have a dual texture design such that the anterior portion 119 has a smooth or polished texture or finish and the posterior portion 121 has a frosted or coarse texture or finish.

In some embodiments, the smooth or polished texture or finish extends along part of the length of the haptic 104 along the anterior side of the haptic 104. In these and other embodiments, the frosted or coarse texture or finish extends along part of the length of the haptic 104 along the posterior side of the haptic 104.

As previously discussed, laser light 125 can be directed at either the lumen filler 126 or the lumen expander 128 disposed within the haptic 104. The laser light 125 can cause a shape change in either the lumen filler 126 or the lumen expander 128 to cause the base power of the optic portion 102 to change. One technical problem faced by the applicant is how to design an intraocular lens such that errant laser energy directed at the haptic(s) of the intraocular lens does not inadvertently harm the patient. One technical solution discovered and developed by the applicant is the IOL 100 disclosed herein comprising a dual texture haptic 104 where the surface texture of an anterior portion of the haptic 104 is substantially smooth or polished to allow the laser light 125 to enter the haptic 104 without substantially interfering with the laser light 125 and the surface texture of a posterior portion of the haptic 104 is substantially frosted or coarse to serve as a safety feature to diffuse the laser light 125.

In some embodiments, the fluid within the optic fluid chamber 108 and the haptic lumen(s) 106 can be an oil. More specifically, in certain embodiments, the fluid within the optic fluid chamber 108 and the haptic lumen(s) 106 can be a silicone oil or fluid. For example, the fluid can be a silicone oil made in part of a diphenyl siloxane. In other embodiments, the fluid can be a silicone oil made in part of a ratio of two dimethyl siloxane units to one diphenyl siloxane unit. More specifically, in some embodiments, the fluid can be a silicone oil made in part of diphenyltetramethyl cyclotrisiloxane or a copolymer of diphenyl siloxane and dimethyl siloxane. In further embodiments, the fluid can be a silicone oil comprising branched polymers.

The fluid (e.g., the silicone oil) can be index matched with a lens body material used to make the optic portion 102. When the fluid is index matched with the lens body material, the entire optic portion 102 containing the fluid can act as a single lens. For example, the fluid can be selected so that it has a refractive index of between about 1.48 and 1.53 (or between about 1.50 and 1.53). In some embodiments, the fluid (e.g., the silicone oil) can have a polydispersity index of between about 1.2 and 1.3. In other embodiments, the fluid (e.g., the silicone oil) can have a polydispersity index of between about 1.3 and 1.5. In other embodiments, the fluid (e.g., the silicone oil) can have a polydispersity index of between about 1.1 and 1.2. Other example fluids are described in U.S. Patent Publication No. 2018/0153682, which is herein incorporated by reference in its entirety.

The optic portion 102 can be made in part of a deformable or flexible material. In some embodiments, the optic portion 102 can be made in part of a deformable or flexible polymeric material. For example, the anterior element 130, the posterior element 132, or a combination thereof can be made in part of a deformable or flexible polymeric material. The one or more haptics 104 (e.g., the first haptic 104A, the second haptic 104B, or a combination thereof) can be made in part of the same deformable or flexible material as the optic portion 102. In other embodiments, the one or more haptics 104 can be made in part of different materials from the optic portion 102.

In some embodiments, the optic portion 102 can comprise or be made in part of a lens body material. The lens body material can be made in part of a cross-linked copolymer comprising a copolymer blend. The copolymer blend can comprise an alkyl acrylate or methacrylate, a fluoro-alkyl (meth)acrylate, and a phenyl-alkyl acrylate. It is contemplated by this disclosure and it should be understood by one of ordinary skill in the art that these types of acrylic cross-linked copolymers can be generally copolymers of a plurality of acrylates, methacrylates, or a combination thereof and the term “acrylate” as used herein can be understood to mean acrylates, methacrylates, or a combination thereof interchangeably unless otherwise specified. The cross-linked copolymer used to make the lens body material can comprise an alkyl acrylate in the amount of about 3% to 20% (wt %), a fluoro-alkyl acrylate in the amount of about 10% to 35% (wt %), and a phenyl-alkyl acrylate in the amount of about 50% to 80% (wt %). In some embodiments, the cross-linked copolymer can comprise or be made in part of an n-butyl acrylate as the alkyl acrylate, trifluoroethyl methacrylate as the fluoro-alkyl acrylate, and phenylethyl acrylate as the phenyl-alkyl acrylate. More specifically, the cross-linked copolymer used to make the lens body material can comprise n-butyl acrylate in the amount of about 3% to 20% (wt %) (e.g., between about 12% to 16%), trifluoroethyl methacrylate in the amount of about 10% to 35% (wt %) (e.g., between about 17% to 21%), and phenylethyl acrylate in the amount of about 50% to 80% (wt %) (e.g., between about 64% to 67%).

The final composition of the cross-linked copolymer used to make the lens body material can also comprise a cross-linker or cross-linking agent such as ethylene glycol dimethacrylate (EGDMA). For example, the final composition of the cross-linked copolymer used to make the lens body material can also comprise a cross-linker or cross-linking agent (e.g., EGDMA) in the amount of about 1.0%. The final composition of the cross-linked copolymer used to make the lens body material can also comprise an initiator or initiating agent (e.g., Perkadox 16) and a UV absorber.

The one or more haptics 104 can comprise or be made in part of a haptic material. The haptic material can comprise or be made in part of a cross-linked copolymer comprising a copolymer blend. The copolymer blend can comprise an alkyl acrylate, a fluoro-alkyl acrylate, and a phenyl-alkyl acrylate. For example, the cross-linked copolymer used to make the haptic material can comprise an alkyl acrylate in the amount of about 10% to 25% (wt %), a fluoro-alkyl acrylate in the amount of about 10% to 35% (wt %), and a phenyl-alkyl acrylate in the amount of about 50% to 80% (wt %). In some embodiments, the cross-linked copolymer used to make the haptic material can comprise n-butyl acrylate in the amount of about 10% to 25% (wt %) (e.g., between about 19% to about 23%), trifluoroethyl methacrylate in the amount of about 10% to 35% (wt %) (e.g., between about 14% to about 18%), and phenylethyl acrylate in the amount of about 50% to 80% (wt %) (e.g., between about 58% to about 62%). The final composition of the cross-linked copolymer used to make the haptic material can also comprise a cross-linker or cross-linking agent, such as EGDMA, in the amount of about 1.0%. The final composition of the cross-linked copolymer used to make the haptic material can also comprise a number of photoinitiators or photoinitiating agents (e.g., camphorquinone, 1-phenyl-1,2-propanedione, and 2-ethylhexyl-4-(dimenthylamino)benzoate).

In some embodiments, the refractive index of the lens body material can be between about 1.48 and about 1.53. In certain embodiments, the refractive index of the lens body material can be between about 1.50 and about 1.53 (e.g., about 1.5178).

The anterior element 130 can be attached or otherwise adhered to the posterior element 132 via adhesives 150 or an adhesive layer. The adhesive layer can be substantially annular-shaped. The adhesives 150 or adhesive layer can be positioned at a peripheral edge of the optic portion 102 in between the anterior element 130 and the posterior element 132. For example, the adhesives 150 can be positioned on top of the raised periphery 144 of the posterior element 132.

The adhesives 150 or adhesive layer can comprise or be made in part of a biocompatible adhesive. The adhesives 150 or adhesive layer can comprise or be made in part of a biocompatible polymeric adhesive.

The adhesives 150 or adhesive layer can comprise or be made in part of a cross-linkable polymer precursor formulation. The cross-linkable polymer precursor formulation can comprise or be made in part of a copolymer blend, a hydroxyl-functional acrylic monomer, and a photoinitiator.

The copolymer blend can comprise an alkyl acrylate (e.g., n-butyl acrylate in the amount of about 41% to about 45% (wt %)), a fluoro-alkyl acrylate (e.g., trifluoroethyl methacrylate in the amount of about 20% to about 24% (wt %)), and a phenyl-alkyl acrylate (phenylethyl acrylate in the amount of about 28% to about 32% (wt %)). The hydroxyl-functional acrylic monomer can be 2-hydroxyethyl acrylate (HEA). The photoinitiator can be used to facilitate curing of the adhesive. For example, the photoinitiator can be Darocur 4265 (a 50/50 blend of diphenyl(2,4,6-trimethylbenzoyl)phosphine oxide and 2-hydroxy2-methylpropiophenone).

In some embodiments, the same adhesives 150 used to bond the anterior element 130 to the posterior element 132 can also be used to bond or affix the one or more haptics 104 to the optic portion 102.

In some embodiments, the composite material can comprise a composite base material, an energy absorbing constituent, and a plurality of expandable components. As previously discussed, one or more portions of each of the haptics 104 can be made of the composite material.

The composite base material can be comprised of hydrophobic acrylic materials. For example, the composite base material can be comprised of phenylethyl acrylate (PEA), a phenylethyl methacrylate (PEMA), or a combination thereof.

In one example embodiment, the composite base material can comprise a methacrylate-functional or methacrylic-functional cross-linkable polymer and reactive acrylic monomer diluents including lauryl methacrylate (n-dodecyl methacrylate or SR313) and ADMA. By controlling the amount of lauryl methacrylate (SR313) to ADMA, the overall corresponding hardness (i.e., more ADMA) or softness (i.e., more SR313) of the cured composite material can be controlled. The methacrylate-functional or methacrylic-functional cross-linkable polymer can be made using the cross-linkable polymer precursor formulation.

The cross-linkable polymer precursor formulation can comprise the same copolymer blend used to make the optic portion and the haptics. The copolymer blend can comprise an alkyl acrylate or methacrylate (e.g., n-butyl acrylate), a fluoro-alkyl (meth)acrylate (e.g., trifluoroethyl methacrylate), and a phenyl-alkyl acrylate (e.g., phenylethyl acrylate). For example, the copolymer blend can comprise n-butyl acrylate in the amount of about 41% to about 45% (wt %), trifluoroethyl methacrylate in the amount of about 20% to about 24% (wt %), and phenylethyl acrylate in the amount of about 28% to about 32% (wt %). The cross-linkable polymer precursor formulation can comprise or be made in part of the copolymer blend, a hydroxyl-functional acrylic monomer (e.g., HEA), and a photoinitiator (e.g., Darocur 4265 or a 50/50 blend of diphenyl(2,4,6-trimethylbenzoyl)-phosphine oxide and 2-hydroxy2-methylpropiophenone).

The composite base material can comprise the methacrylate-functional or methacrylic-functional cross-linkable polymer (as discussed above) in the amount of about 50% to about 65% (e.g., about 55% to about 60%) (wt %), the reactive acrylic monomer diluent lauryl methacrylate (SR313) in the amount of about 32% to about 38% (e.g., about 32.70%) (wt %), the reactive acrylic monomer diluent adamantly methacrylate (ADMA) in the amount of about 5% to about 9% (e.g., about 7.30%) (wt %).

The composite material can be made in several operations. The first operation can comprise preparing an uncolored composite base material. The second operation can comprise mixing the composite base material with an energy absorbing constituent, expandable components, and initiators such as one or more photoinitiators, thermal initiators, or a combination thereof. The third operation can comprise placing the uncured composite material into a desired location within the haptics 104 (e.g., in proximity to the channel 148), and curing the composite material in place.

For example, the uncolored composite base material can be mixed with an energy absorbing constituent such as a dye (e.g., Disperse Red 1 dye) or pigment (graphitized carbon black).

In some embodiments, the expandable components can make up about 5.0% to about 15.0% by weight of a final formulation of the composite material. More specifically, the expandable components can make up about 8.0% to about 12.0% (e.g., about 10.0%) by weight of a final formulation of the composite material. In these and other embodiments, the energy absorbing constituent can make up about 0.044% to about 0.44% (or about 0.55%) by weight of the final formulation of the composite material.

The photoinitiator can be Omnirad 2022 (bis(2,4,6-trimethylbenzoyl)phenyl-phosphineoxide/2-hydroxy-2-methyl-1-phenyl-propan-1-one). The photoinitiator can make up about 1.30% by weight of a final formulation of the composite material. In addition, the composite material can also comprise a thermal initiator. The thermal initiator can make up about 1.00% by weight of a final formulation of the composite material. In some embodiments, the thermal initiator can be a dialkyl peroxide such as Luperox® peroxide. In other embodiments, the thermal initiator can be Perkadox.

In some embodiments, the energy absorbing constituent can absorb the external energy (e.g., laser energy), convert the energy to heat, and conduct the energy to the composite base material to expand the composite base material.

In some embodiments, the expandable components can be expandable microspheres comprising an expandable thermoplastic shell and a blowing agent contained within the expandable thermoplastic shell. The microspheres can be configured to expand such that a diameter of at least one of the microspheres can increase about 2× the original diameter. In other embodiments, the microspheres can be configured to expand such that the diameter of at least one of the microspheres can increase about 4× or four times the original diameter. further embodiments, the microspheres can be configured to expand such that the diameter of at least one of the microspheres can increase between about 2× and about 4× (or about 3.5λ) the original diameter. For example, the microspheres can have a diameter of about 12 μm at the outset. In response to an external energy applied or directed at the composite material or in response to energy transferred or transmitted to the microspheres, the diameter of the microspheres can increase to about 40 μm.

The volume of at least one of the microspheres can be configured to expand between about ten times (10×) to about 50 times (50×) in response to the external energy applied or directed at the composite material or in response to energy transferred or transmitted to the microspheres.

In some embodiments, the blowing agent can be an expandable fluid, such as an expandable gas. More specifically, the blowing agent can be a branched-chain hydrocarbon. For example, the blowing agent can be isopentane. In other embodiments, the blowing agent can be or comprise cyclopentane, pentane, or a mixture of cyclopentane, pentane, and isopentane.

Each of the expandable components can comprise a thermoplastic shell. A thickness of the thermoplastic shell can change as the expandable component increases in size. More specifically, the thickness of the thermoplastic shell can decrease as the expandable component increases in size. For example, when the expandable components are expandable microspheres, the thickness of the thermoplastic shell (i.e., its thickness in a radial direction) can decrease as the diameter of the expandable microsphere increases.

In some embodiments, the thermoplastic shell can be made in part of nitriles or acrylonitrile copolymers. For example, the thermoplastic shell can be made in part of acrylonitrile, styrene, butadiene, methyl acrylate, or a combination thereof.

As previously discussed, the expandable components can make up between about 8.0% to about 12% by weight of a final formulation of the composite material. The expandable components can make up about 10% by weight of a final formulation of the composite material.

The expandable components can be dispersed or otherwise distributed within the composite base material making up the bulk of the composite material. The composite base material can serve as a matrix for holding or carrying the expandable components. The composite material can expand in response to an expansion of the expandable components (e.g., the thermoplastic microspheres). For example, a volume of the composite material can increase in response to the expansion of the expandable components.

The composite material also comprises an energy absorbing constituent. In some embodiments, the energy absorbing constituent can be an energy absorbing colorant.

In certain embodiments, the energy absorbing colorant can be an energy absorbing dye. For example, the energy absorbing dye can be an azo dye. In some embodiments, the azo dye can be a red azo dye such as Disperse Red 1 dye. In other embodiments, the azo dye can be an orange azo dye such as Disperse Orange dye (e.g., Disperse Orange 1), a yellow azo dye such as Disperse Yellow dye (e.g., Disperse Yellow 1), a blue azo dye such as Disperse Blue dye (e.g., Disperse Blue 1), or a combination thereof.

In additional embodiments, the energy absorbing colorant can be or comprise a pigment. For example, the energy absorbing colorant can be or comprise graphitized carbon black as the pigment.

Similar to the expandable components, the energy absorbing constituent can be dispersed or otherwise distributed within the composite base material making up the bulk of the composite material. The composite base material can serve as a matrix for holding or carrying the expandable components and the energy absorbing constituent.

As previously discussed, the energy absorbing constituent can make up between about 0.025% to about 1.0% (or, more specifically, about 0.045% to about 0.45%) by weight of a final formulation of the composite material.

The energy absorbing constituent (e.g., azo dye, graphitized carbon black, or a combination thereof) can absorb or capture an external energy (e.g., light energy or, more specifically, laser light) applied or directed at the composite material. The energy absorbing constituent can absorb or capture the external energy and then transform or transfer the energy into thermal energy or heat to the expandable components.

The thermoplastic shell can soften and begin to flow as thermal energy is transferred or transmitted to expandable components. The thermoplastic shell of the expandable components can then begin to thin or reduce in thickness in response to the thermal energy transferred or transmitted to the expandable components. As the thermoplastic shell begins to soften and reduce in thickness, the blowing agent within the expandable components can expand. The blowing agent can also expand in response the thermal energy or heat transferred or transmitted to the expandable components. Expansion of the blowing agents can cause the expandable components (e.g., the thermoplastic microspheres) to expand or increase in volume. This ultimately causes the composite material to expand or increase in volume.

As previously discussed, the external energy can be laser light 125 and the energy absorbing constituent can absorb or capture the laser light 125 directed at the composite material and transform or transfer the light energy into thermal energy or heat to the expandable components. The blowing agent within the expandable components can expand or become energized in response to the thermal energy or heat. The expandable components and, ultimately, the composite material can expand or increase in volume in response to this light energy directed at the composite material.

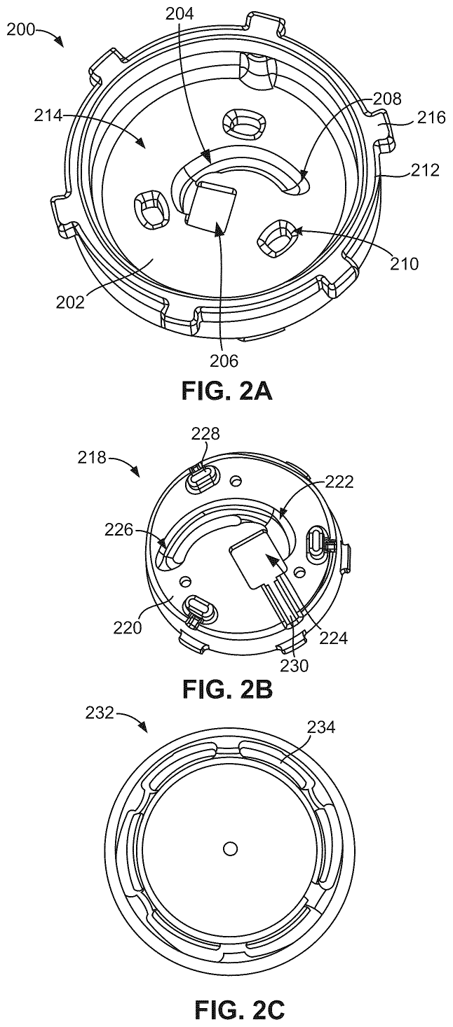

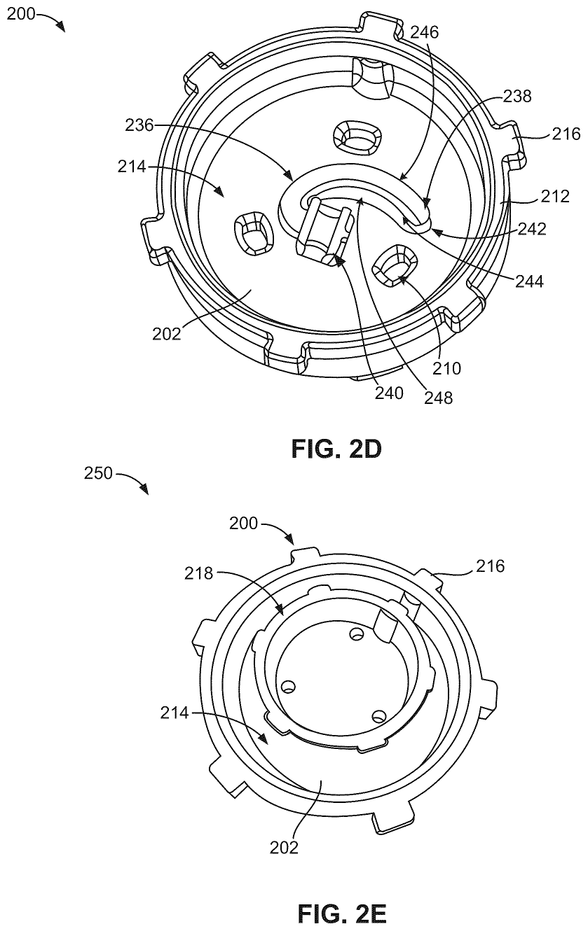

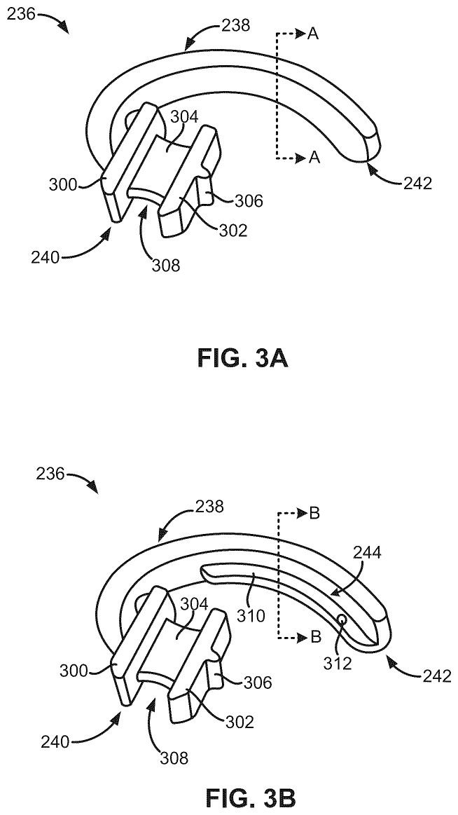

FIG. 2A illustrates an embodiment of a first mold component 200 comprising a first molding surface 202 defined by a first partial component cavity 204. The first partial component cavity 204 can be an indentation or depression defined along the first molding surface 202. The first partial component cavity 204 can be shaped, sized, or otherwise configured to accommodate part of a soluble core component 236 (see FIGS. 2D and 3A-3B). In some embodiments, the first mold component 200 can also be referred to as a wafer mold.

In some embodiments, the first partial component cavity 204 can be connected to or be contiguous with a first partial positioning cavity 206. For example, the first partial component cavity 204 can be in fluid communication with the first partial positioning cavity 206. In other embodiments, the first partial component cavity 204 can be positioned in proximity to a first partial positioning cavity 206. For example, the first partial component cavity 204 can be near the first partial positioning cavity 206 but not directly connected to the first partial positioning cavity 206.

The first partial component cavity 204 can be shaped, sized, or otherwise configured to accommodate at least part of a core body 238 (see FIGS. 2D and 3A-3B) of the soluble core component 236. For example, the first partial component cavity 204 can be shaped, sized, or otherwise configured to accommodate a posterior half of the core body 238 of the soluble core component 236.

The first partial positioning cavity 206 can be shaped, sized, or otherwise configured to accommodate at least part of a positioning piece 240 (see FIGS. 2D and 3A-3B) of the soluble core component 236. For example, the first partial positioning cavity 206 can be shaped, sized, or otherwise configured to accommodate a posterior half of the positioning piece 240 of the soluble core component 236.

Although FIGS. 2A and 2D illustrate the first mold component 200 comprising one first partial positioning cavity 206, it is contemplated by this disclosure that the first mold component 200 can comprise a plurality of first partial positioning cavities 206 for accommodating multiple positioning pieces 240.

In some embodiments, the first partial component cavity 204 can terminate at a first cavity distal end 208. The first cavity distal end 208 can be positioned at a distance from the first partial positioning cavity 206. The first cavity distal end 208 can be positioned closer to the first mold component wall 212 than the first partial positioning cavity 206. In these embodiments, the first cavity distal end 208 is not in contact with the first mold component wall 212.

In certain embodiments, the first cavity distal end 208 can be rounded or curved. For example, the first cavity distal end 208 can be substantially shaped as a partial ovoid or ellipsoid. The first cavity distal end 208 can be substantially shaped as a partial sphere.

In other embodiments not shown in FIG. 2A but contemplated by this disclosure, the first partial component cavity 204 can be connected to another partial positioning cavity instead of terminating at a closed-off distal end. In these embodiments, the first partial component cavity 204 can be disposed in between two partial positioning cavities.

As shown in FIG. 2A, the first mold component 200 can also comprise a plurality of leg cavities 210 defined along the first molding surface 202. The leg cavities 210 can be divots or indentations configured to accommodate legs or other support structures extending from a complementary mold component (for example, a second mold component 218, see FIG. 2B).

The leg cavities 210 can surround or be positioned radially outward of the first partial component cavity 204. The leg cavities 210 can be arranged in a particular pattern or positioned in a specific manner to allow a complementary mold component (for example, the second mold component 218) to be registered to the first mold component 200. For example, the leg cavities 210 can be arranged in a particular pattern or aligned in a specific manner to allow the first partial component cavity 204 to be matched or otherwise aligned with a second partial component cavity 222 on the second mold component (see, for example, FIG. 2B). The first partial component cavity 204 can be properly aligned with the second partial component cavity 222 when the legs or other support structures on the second mold component 218 are inserted or placed into the leg cavities 210 of the first mold component 200.

In the example embodiment shown in FIG. 2A, the leg cavities 210 can be arranged in a substantially triangular pattern. In other embodiments not shown in the figures but contemplated by this disclosure, the leg cavities 210 can be arranged in a circular pattern, a diamond or rhombus pattern, a square pattern, or another polygonal pattern.

The first molding surface 202 can be sunken or recessed with respect to a first mold component wall 212. The sunken or recessed first molding surface 202 can create a reservoir space 214 within the first mold component 200. The reservoir space 214 can receive a lens component material when the entire mold is assembled.

As shown in FIG. 2A, in one embodiment, the first mold component 200 can be configured as a circular cap or circular mold-half. In this embodiment, the first mold component wall 212 can be substantially shaped as the lateral sides of a cylinder. Moreover, the first molding surface 202 can also be substantially circular-shaped.

In other embodiments, the first mold component 200 can be configured as a rectangular cap or rectangular mold-half. In these embodiments, the first mold component wall 212 can be substantially shaped as the lateral sides of a cuboid. Moreover, the first molding surface 202 can also be substantially rectangular-shaped.

In further embodiments, the first mold component 200 including the first molding surface 202 and the first mold component wall 212 can be oval-shaped or ellipsoid-shaped. In additional embodiments, the first mold component 200 including the first molding surface 202 and the first mold component wall 212 can be triangular-shaped or shaped as another type of polygon.

The first mold component 200 can also comprise a plurality of engagement features 216 extending radially outward from the first mold component wall 212. In some embodiments, the engagement features 216 can be tabs or flaps extending radially outward from the first mold component wall 212. In other embodiments, the engagement features 216 can comprise threads or a thread-pattern defined on an exterior of the first mold component wall 212. The engagement features 216 can allow the first mold component 200 to more easily engage or couple with a clamping member 232 (see, for example, FIG. 2C) for securing or holding together an assembled mold.

FIG. 2B illustrates an embodiment of a second mold component 218 comprising a second molding surface 220 defined by a second partial component cavity 222. The second partial component cavity 222 can be an indentation or depression defined along the second molding surface 220. The second partial component cavity 222 can be shaped, sized, or otherwise configured to accommodate part of the soluble core component 236. In some embodiments, the second mold component 218 can also be referred to as a wafer mold.

The second molding surface 220 can be configured or otherwise designed to face the first molding surface 202 when the second mold component 218 is mated to the first mold component 200 to form an assembled mold 250 (see FIG. 2E). The second molding surface 220 can be located on an underside of the second mold component 218 (the underside is facing upwards in FIG. 2B).

In some embodiments, the second partial component cavity 222 can be connected to or be contiguous with a second partial positioning cavity 224. For example, the second partial component cavity 222 can be in fluid communication with the second partial positioning cavity 224. In other embodiments, the second partial component cavity 222 can be positioned in proximity to a second partial positioning cavity 224. For example, the second partial component cavity 222 can be near the second partial positioning cavity 224 but not directly connected to the second partial positioning cavity 224.

The second partial component cavity 222 can be shaped, sized, or otherwise configured to accommodate at least part of the core body 238 of the soluble core component 236. For example, the second partial component cavity 222 can be shaped, sized, or otherwise configured to accommodate an anterior half of the core body 238 of the soluble core component 236.

In addition, the second partial positioning cavity 224 can be shaped, sized, or otherwise configured to accommodate at least part of a positioning piece 240 of the soluble core component 236. For example, the second partial positioning cavity 224 can be shaped, sized, or otherwise configured to accommodate a posterior half of the positioning piece 240 of the soluble core component 236.

Although FIG. 2B illustrates the second mold component 218 comprising one second partial positioning cavity 224, it is contemplated by this disclosure that the second mold component 218 can comprise a plurality of second partial positioning cavities 224 for accommodating multiple positioning pieces 240.

In some embodiments, the second partial component cavity 222 can terminate at a second cavity distal end 226. The second cavity distal end 226 can be positioned at a distance from the second partial positioning cavity 224. The second cavity distal end 226 can be positioned closer to the edge or periphery of the second molding surface 220 than the second partial positioning cavity 224. In these embodiments, the second cavity distal end 226 does not reach the edge of the second molding surface 220.

In certain embodiments, the second cavity distal end 226 can be rounded or curved. For example, the second cavity distal end 226 can be substantially shaped as a partial ovoid or ellipsoid. The second cavity distal end 226 can be substantially shaped as a partial sphere.

In other embodiments not shown in FIG. 2B but contemplated by this disclosure, the second partial component cavity 222 can be connected to another partial positioning cavity instead of terminating at a closed-off distal end. In these embodiments, the second partial component cavity 222 can be disposed in between two partial positioning cavities.

The second mold component 218 can also comprise a plurality of legs 228 or support structures protruding or otherwise extending from the second molding surface 220. In some embodiments, the legs 228 can protrude or extend substantially orthogonally or perpendicularly from the second molding surface 220. The legs 228 of the second mold component 218 can fit within the leg cavities 210 of the first mold component 200 when the second mold component 218 is mated or otherwise coupled to the first mold component 200.

As shown in FIG. 2B, the legs 228 can surround or be positioned radially outward of the second partial component cavity 222. The legs 228 can be arranged in a particular pattern or aligned in a specific manner to allow the second mold component 218 to be properly registered to the first mold component 200. For example, the legs 228 can be arranged in a particular pattern or aligned in a specific manner to allow the first partial component cavity 204 to be matched or otherwise aligned with the second partial component cavity 222. The first partial component cavity 204 can be properly aligned with the second partial component cavity 222 when the legs 228 on the second mold component 218 are inserted or placed into the leg cavities 210 of the first mold component 200.

In the example embodiment shown in FIG. 2B, the legs 228 can be arranged in a substantially triangular pattern. In other embodiments not shown in the figures but contemplated by this disclosure, the legs 228 can be arranged in a circular pattern, a diamond or rhombus pattern, a square pattern, or another polygonal pattern.

As shown in FIG. 2B, in one embodiment, the second mold component 218 can be configured as a circular mold-half. In this embodiment, the second molding surface 220 can also be substantially circular-shaped.

In other embodiments, the second mold component 218 can be configured as a rectangular mold-half. In these embodiments, the second molding surface 220 can also be substantially rectangular-shaped.

In further embodiments, the second mold component 218 including the second molding surface 220 can be oval-shaped or ellipsoid-shaped. In additional embodiments, the second mold component 218 including the second molding surface 220 can be triangular-shaped or shaped as another type of polygon.

In some embodiments, the second mold component 218 can be sized to fit within the reservoir space 214 of the first mold component 200. At least part of the second mold component 218 can be within the reservoir space 214 when the first mold component 200 is mated or otherwise coupled to the second mold component 218.

The first partial component cavity 204 and the second partial component cavity 222 can come together to form a component mold cavity when the first mold component 200 is mated or otherwise coupled to the second mold component 218. For example, the first mold component 200 can be mated to the second mold component 218 when the first mold component 200 is clamped to the second mold component 218. In addition, the first partial positioning cavity 206 and the second partial positioning cavity 224 can come together to form a positioning cavity when the first mold component 200 is mated or otherwise coupled to the second mold component 218.

The component mold cavity can be in fluid communication with the reservoir space 214 of the first mold component 200. The lens component material can seep into the component mold cavity from the reservoir space 214. The low viscosity lens component material can seep into the component mold cavity through the parting lines between the first mold component 200 and the second mold component 218. The lens component material can be drawn into the component mold cavity from the reservoir space 214 when the assembled mold 250 is placed into a vacuum chamber and a vacuum is drawn.

The second mold component 218 can also comprise a vent 230 or opening connecting the second partial positioning cavity 224 to an edge or periphery of the second mold component 218. The vent 230 can allow air to escape while the component mold cavity is being filled with the low-viscosity lens component material.

In some embodiments, the lens component material can be the haptic material disclosed in the preceding sections. For example, the lens component material can comprise curable acrylic monomers. The lens component material can be a substantially hydrophobic material.

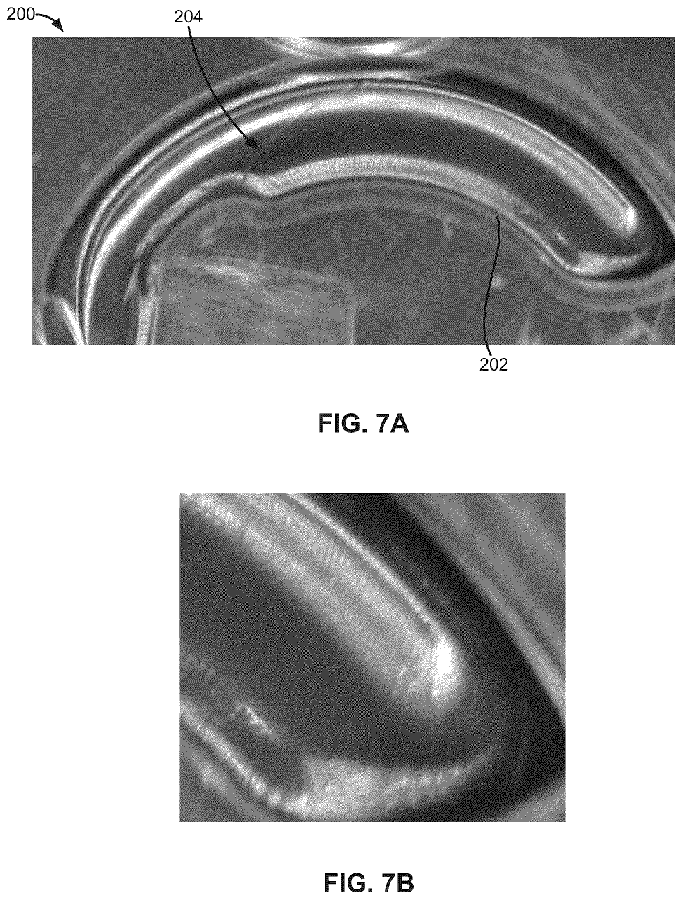

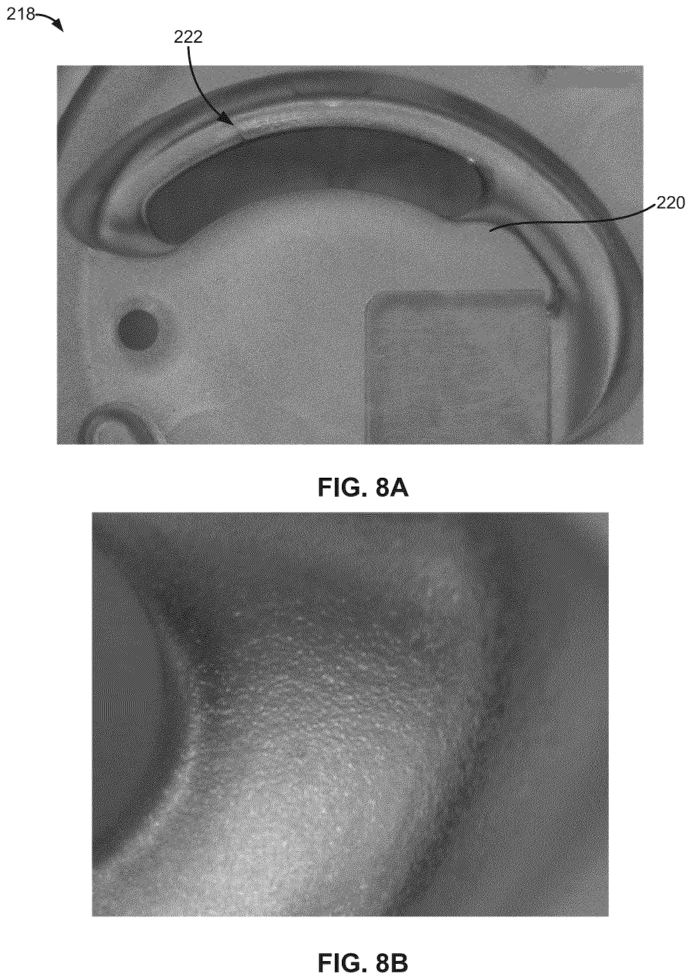

As will be discussed in more detail in relation to FIGS. 7A-7B and 8A-8B, the first molding surface 202 (including the first partial component cavity 204) of the first mold component 200 can have a first surface roughness. Moreover, the second molding surface 220 (including the second partial component cavity 222) of the second mold component 218 can have a second surface roughness. The second surface roughness can be greater (i.e., more rough or more coarse) than the first surface roughness such that at least part of the first molding surface 202 is smoother or more polished than the second molding surface 220.

Since the first mold component 200 and the second mold component 218 are used to make the haptic 104, the first molding surface 202 can impart a surface texture (including a surface roughness) to an anterior side of the haptic 104 and the second molding surface 220 can impart a surface texture (including a surface roughness) to a posterior side of the haptic 104.

In some embodiments, the first molding surface 202 of the first mold component 200 can impart a smooth or polished texture or finish to at least part of the haptic anterior portion 119. In these and other embodiments, the second molding surface 220 of the second mold component 218 can impart a frosted or coarse texture to at least part of the haptic posterior portion 121.

One technical problem faced by the applicant is how to make an intraocular lens with a dual texture haptic. One technical solution discovered and developed by the applicant are the mold components disclosed herein including the first mold component 200 comprising the first molding surface 202 and the second mold component 218 comprising the second molding surface 220 where the surface roughness (as measured in either roughness average (Ra) and/or Society of the Plastics Industry (SPI) grade) of the second molding surface 220 is greater than the surface roughness of the first molding surface 202.

FIG. 2C illustrates an embodiment of a clamping member 232 for securing the first mold component 200 to the second mold component 218. In the example embodiment shown in FIG. 2C, the clamping member 232 can be a clamping cap configured to be detachably fastened to the top of the first mold component 200 when the second mold component 218 is mated to the first mold component 200.

As previously discussed, the first mold component 200 can comprise a plurality of engagement features 216 extending radially outward from the first mold component wall 212. In some embodiments, the engagement features 216 can be tabs or flaps extending radially outward from the first mold component wall 212. The clamping member 232 can comprise a plurality of grooves 234 or notches for interlocking with or otherwise receiving the engagement features 216.

In other embodiments, the engagement features 216 can comprise threads or a thread-pattern defined on an exterior of the first mold component wall 212. In these embodiments, the clamping member 232 can comprise a complementary thread-pattern to allow the clamping member 232 to be screwed on to the first mold component 200.

In further embodiments, the clamping member 232 can be a traditional clamp or vise-type device for tightly securing the first mold component 200 to the second mold component 218.

The clamping member 232 can tightly secure the first mold component 200 to the second mold component 218 to prevent the various mold parts from shifting or moving relative to one another. The clamping member 232 can tightly secure the first mold component 200 to the second mold component 218 during the curing step to prevent the lens component material from seeping out from mold parting lines. The clamping member 232 can be coupled to the first mold component 200 to form an enclosed chamber. Although not shown in FIG. 2C, when the clamping member 232 is implemented as a clamping cap, the clamping cap can have a septum or inlet port defined along a surface (for example, a top surface) of the clamping cap. An injection nozzle or another delivery device can be inserted through the septum or inlet port to inject or otherwise introduce the curable lens component material into the reservoir space 214 within the enclosed chamber.

In some embodiments, the clamping member 232 can be made in part of a polymeric material, a metallic material, or a combination thereof. In further embodiments, the clamping member 232 can be made in part of a ceramic material. The clamping member 232 can be unfastened or uncoupled from the first mold component 200 after the curing step. The clamping member 232 can be unfastened or uncoupled from the first mold component 200 prior to separating the first mold component 200 from the second mold component 218.

FIG. 2D illustrates that at least part of a soluble core component 236 can be placed on the first molding surface 202 of the first mold component 200. Although FIG. 2D illustrates the soluble core component 236 placed on the first molding surface 202, it is contemplated by this disclosure that the soluble core component 236 can also be initially placed on the second molding surface 220 of the second mold component 218. In the following sections, any references to placement or positioning of the soluble core component 236 on the first molding surface 202 of the first mold component 200 can also refer to placement or positioning of the soluble core component 236 on the second molding surface 220 of the second mold component 218.

In cases where the lens component formed is a haptic (for example, any of the haptics 104 shown in FIGS. 1A-1C), the soluble core component 236 can comprise a core body 238 connected to at least one positioning piece 240. In these embodiments, the core body 238 can terminate at a core distal end 242.

At least part of the positioning piece 240 of the soluble core component 236 can be pressed or inserted into the first partial positioning cavity 206 of the first mold component 200. The positioning piece 240 can be configured to fit tightly within the first partial positioning cavity 206 when the positioning piece 240 is pressed or inserted into the first partial positioning cavity 206. The soluble core component 236 can be detachably coupled to the first mold component 200 when the positioning piece 240 is pressed or inserted into the first partial positioning cavity 206.

In some embodiments, the positioning piece 240 of the soluble core component 236 can be the only portion of the soluble core component 236 that physically contacts the first molding surface 202. For example, the positioning piece 240 of the soluble core component 236 can be in physical contact with the sunken or recessed portions of the first molding surface 202 defining the first partial positioning cavity 206. In these embodiments, the remainder of the soluble core component 236 coupled to or extending from the positioning piece 240 (e.g., the curved core body 238 and the core distal end 242) can be positioned over the first molding surface 202 but not be in physical contact with the first molding surface 202 when the soluble core component 236 is detachably coupled to the first mold component 200. For example, the core body 238 and the core distal end 242 can be raised or elevated by the positioning piece such that no part of the core body 238 and core distal end 242 physically contacts the first molding surface 202.

In these embodiments, at least part of the core body 238 and the core distal end 242 can be positioned within the first partial component cavity 204 but not touch any part of the first molding surface 202 defining or serving as the walls and bottom of the first partial component cavity 204 when the positioning piece 240 is pressed or inserted into the first partial positioning cavity 206. In addition, no part of the core body 238 of the soluble core component 236 extends past the contour or boundary of the first partial component cavity 204 when the positioning piece 240 is pressed or inserted into the first partial positioning cavity 206. Moreover, the first cavity distal end 208 can extend past or extend beyond the core distal end of the soluble core component 236 when the positioning piece 240 is pressed or inserted into the first partial positioning cavity 206. Furthermore, the raised first mold component wall 212 can surround the soluble core component 236 when the positioning piece 240 is pressed or inserted into the first partial positioning cavity 206.

FIG. 2D also illustrates that the core body 238 of the soluble core component 236 can comprise a radially inner body portion 244 and a radially outer body portion 246. The radially inner body portion 244 can be the side of the core body 238 facing toward or in proximity to the positioning piece 240 when the positioning piece 240 is pressed or inserted into the first partial component cavity 204 and the core body 238 is partially positioned within the first partial component cavity 204. The radially outer body portion 246 can be the side of the core body 238 facing away from the positioning piece 240 when the positioning piece 240 is pressed or inserted into the first partial component cavity 204 and the core body 238 is partially positioned within the first partial component cavity 204. The radially inner body portion 244 can be separated from an inner edge 248 of the first partial component cavity 204 by a radially inner separation distance. The inner edge 248 of the first partial component cavity 204 can be part of the inner contour or boundary of the first partial component cavity 204. The radially outer body portion 246 can be separated from an outer edge (not shown in FIG. 2D) of the first partial component cavity 204 by a radially outer separation distance. The outer edge of the first partial component cavity 204 can be part of the outer contour or boundary of the first partial component cavity 204.

In some embodiments, the radially inner separation distance can be greater than the radially outer separation distance. This disparity in separation distances can allow more of the lens component material to occupy the space separating the radially inner body portion 244 from the inner edge 248 than the space separating the radially outer body portion 246 from the outer edge of the first partial component cavity 204. This can allow the haptic (see, for example, the haptic 104 of FIGS. 1B and 1C) to be formed with a thicker radially inner portion relative to the radially outer portion of the haptic.

The unique positioning of the soluble core component 236 relative to the mold component(s) can allow the lens component material (e.g., the haptic material) to enter and fill the space surrounding the soluble core component 236 within the component cavities. The haptic material can then be cured to form a molded haptic surrounding the soluble core component 236. After the curing process, the molded haptic, including the soluble core component 236 within the molded haptic, can then be immersed in a solvent to dissolve the soluble core component 236. Once the soluble core component 236 is dissolved, the space within the molded haptic previously occupied by the soluble core component 236 can serve as a haptic fluid chamber or haptic lumen (see, for example, the haptic lumen 106 of haptic 104).

FIG. 2E illustrates that an assembled mold 250 can be formed by mating the second mold component 218 to the first mold component 200. The soluble core component 236 can be pressed or otherwise placed into position within the first mold component 200 prior to the second mold component 218 mating with the first mold component 200. The soluble core component 236 can be placed into position within the first mold component 200 by pressing or inserting the positioning piece 240 of the soluble core component 236 into the first partial positioning cavity 206 and positioning at least part of the core body 238 of the soluble core component 236 within the first partial component cavity 204. As previously discussed, the soluble core component 236 can also be initially pressed or otherwise placed into position within the second mold component 218 prior to the two mold components mating together. The soluble core component 236 can be disposed in between the first molding surface 202 and the second molding surface 220 when the assembled mold 250 is formed.

Mating the second mold component 218 to the first mold component 200 can comprise inserting the legs 228 of the second mold component 218 into the leg cavities 210 of the first mold component 200 and pressing the second mold component 218 against the first mold component 200. As previously discussed, the legs 228 of the second mold component 218 and the leg cavities 210 of the first mold component 200 can be arranged such that the first partial component cavity 204 of the first mold component 200 fits precisely over the second partial component cavity 222 of the second mold component 218. For example, the contours or boundaries of the first partial component cavity 204 can be aligned with the contours or boundaries of the second partial component cavity 222 when the second mold component 218 is mated to the first mold component 200. Moreover, the contours or boundaries of the first partial positioning cavity 206 can also be aligned with the contours or boundaries of the second partial positioning cavity 224 when the second mold component 218 is mated with the first mold component 200.

The first partial component cavity 204 and the second partial component cavity 222 can form a component mold cavity when the assembled mold 250 is formed by mating the second mold component 218 to the first mold component 200. The core body 238 of the soluble core component 236 can be disposed within the component mold cavity when the second mold component 218 is mated to the first mold component 200. The positioning piece 240 of the soluble core component 236 can be disposed outside of the component mold cavity when the second mold component 218 is mated to the first mold component 200.

FIG. 2E illustrates that the second mold component 218 can fit within the reservoir space 214 of the first mold component 200 when the second mold component 218 is mated with the first mold component 200. A curable lens component material (for example, the haptic material) can be introduced (e.g., injected, poured, etc.) into the reservoir space 214 once the assembled mold 250 has been formed. In some embodiments, the low-viscosity lens component material can seep into the component mold cavity through the parting lines between the first mold component 200 and the second mold component 218 when the mold components are mated together. In other embodiments, the lens component material can enter into the component mold cavity through other openings or conduits defined along the second molding surface 220, the first molding surface 202, or a combination thereof.

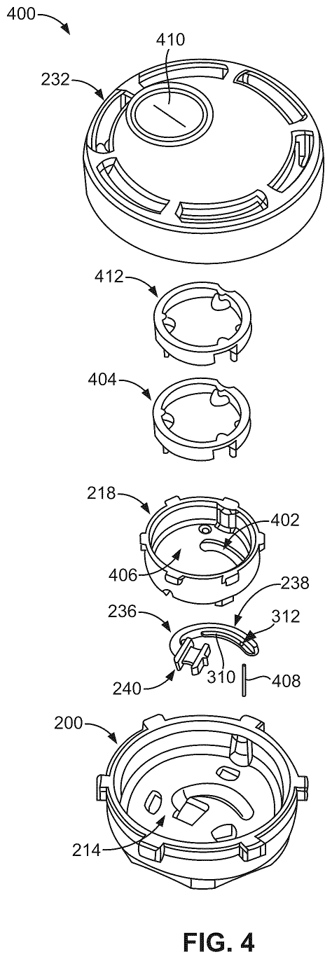

As previously discussed, a clamping member 232 (see, for example, FIG. 2C or FIG. 4) can be detachably coupled or fastened to the first mold component 200 after the first mold component 200 is mated to the second mold component 218. For example, the clamping member 232 can be a clamping cap configured to be detachably fastened to the top of the first mold component 200 when the second mold component 218 is mated to the first mold component 200. The clamping cap can create an enclosed chamber and press the second mold component 218 tightly against the first mold component 200.

A curable lens component material can then be introduced into this enclosed chamber. For example, the lens component material can be injected or otherwise introduced into the reservoir space 214 through a septum 410 (see FIG. 4) or other opening defined along the clamping member 232. Once the lens component material has been introduced, the assembled mold 250, including the clamping member 232 coupled to the assembled mold 250, can be placed within a vacuum chamber. A vacuum can be pulled after the assembled mold 250 is placed within the vacuum chamber to draw the lens component material into the component mold cavity. The low-viscosity lens component material can seep into the component mold cavity through parting lines or miniscule gaps in between the first mold component 200 and the second mold component 218. While under vacuum, the lens component material can fill the space within the assembled mold 250 unoccupied by the soluble core component 236. For example, the lens component material can fill the space within the component mold cavity unoccupied by the core body 238 of the soluble core component 236.

Once the lens component material has filled the component mold cavity, the assembled mold 250 comprising the lens component material can be cured in a curing chamber. In some embodiments, the assembled mold 250 can be cured using light such as ultraviolet (UV) light or blue light.

More specifically, in some embodiments, the assembled mold 250 can be cured in a UV light chamber. For example, the assembled mold 250 can be cured using a UV-generating xenon lamp or xenon-mercury lamp. For example, the assembled mold 250 can be cured for between 30 minutes and 60 minutes under UV light. In other embodiments, the assembled mold 250 can be cured using blue light such as light emitted by high-intensity blue light-emitting diode (LED) lights. For example, the assembled mold 250 can be cured for between 10 hours and 12 hours under blue light. In other embodiments, the assembled mold 250 can also be cured using heat or a combination of light and heat.

The clamping member 232 can clamp the first mold component 200 to the second mold component 218 during the curing procedure. The lens component material can be cured into a molded lens component such as a haptic.

In some embodiments, both the first mold component 200 and the second mold component 218 can be made of a non-soluble material. The non-soluble material can be a material not capable of dissolving within a solvent (e.g., water) in a practicable amount of time. For example, the first mold component 200 and the second mold component 218 can be made of a non-soluble polymeric material. In some embodiments, the first mold component 200 and the second mold component 218 can be made from thermoplastic resins including polyolefins such as polymethylpentene (PMP), polypropylene (PP), medium or high density polyethylene (PE), polystyrene, or copolymers thereof.

The first mold component 200 and the second mold component 218 can also be made from polyacetal resins, polyacrylethers, polyarylether sulfones, different types of nylon including nylon 6, nylon 66 and nylon 11.

As a more specific example, the first mold component 200, the second mold component 218, or a combination thereof can be made in part of a 4-methyl-1-pentene-based olefin copolymer such as TPX™ polymethyl pentene distributed by Mitsui Chemicals, Inc.

In other embodiments, the first mold component 200 and the second mold component can be made of a corrosion-resistant metallic material such as stainless steel.

In instances where both the first mold component 200 and the second mold component 218 are made of a non-soluble material, the method of forming the lens component (e.g., the haptic) can comprise separating the first mold component 200 from the second mold component 218 after the curing step and removing the molded lens component from either the first mold component 200 or the second mold component 218. At least a segment of the soluble core component 236 can be within the molded lens component or partially encapsulated by the molded lens component when the molded lens component is removed from the first mold component 200 or the second mold component 218. For example, the core body 238 of the soluble core component 236 can be within the molded lens component or partially encapsulated by the molded lens component when the molded lens component is removed from the first mold component 200 or the second mold component 218. Moreover, the positioning piece 240 of the soluble core component 236 can be connected to the molded lens component but not contained or encapsulated within the molded lens component.

The method can further comprise immersing the molded lens component in a solvent to dissolve the soluble core component 236. The method can also comprise heating the solvent to between about 35° C. and 55° C. and agitating the solvent by sonication or stirring to expedite the dissolution of the soluble core component 236. In some embodiments, the soluble core component 236 can be dissolved after being immersed in the solvent between about 6 hours to about 24 hours. What remains after the soluble core component has dissolved is the molded lens component (for example, a haptic having a cavity defined therein).

The soluble core component 236 can be made of a soluble material such as polyvinyl alcohol. In some embodiments, the solvent can be water.

In some embodiments, both the first mold component 200 and the second mold component 218 can be made of a soluble material. In certain embodiments, the first mold component 200 and the second mold component 218 can be made of the same soluble material as the material used to make the soluble core component 236. For example, the first mold component 200, the second mold component 218, and the soluble core component 236 can be made of polyvinyl alcohol. Alternatively, one or both of the first mold component 200 and the second mold component 218 can be made of a different soluble material than the material used to make the soluble core component 236.

In instances where both the first mold component 200 and the second mold component 218 are made of a soluble material, the method of forming the lens component (e.g., the haptic) can comprise immersing the entire assembled mold 250 in a solvent after the curing step. The entire assembled mold 250 can be immersed in the solvent to dissolve the soluble core component 236, the first mold component 200, and the second mold component 218. The method can also comprise heating the solvent to between about 35° C. and 55° C. and agitating the solvent by sonication or stirring to expedite the dissolution of the soluble core component 236, the first mold component 200, and the second mold component 218. In some embodiments, the soluble core component 236, the first mold component 200, and the second mold component 218 can be dissolved after being immersed in the solvent between about 6 hours to about 24 hours. What remains after all of the soluble material has dissolved is the molded lens component (for example, a haptic having a cavity defined therein).

In some embodiments, either the first mold component 200 or the second mold component 218 (but not both) can be made of a soluble material. In these embodiments, the other mold component can be made of a non-soluble material. The non-soluble material can be a material not capable of dissolving within a solvent (e.g., water) in a practicable amount of time.

For example, either the first mold component 200 or the second mold component 218 can be made of the same soluble material as the material used to make the soluble core component 236. More specifically, in some embodiments, the first mold component 200 or the second mold component 218 and the soluble core component 236 can be made of polyvinyl alcohol. Alternatively, either the first mold component 200 or the second mold component 218 can be made of a different soluble material than the material used to make the soluble core component 236. The other mold component in these instances can be made of a non-soluble material. In some embodiments, the first mold component 200 can be made of polyvinyl alcohol and the second mold component 218 can be made from thermoplastic resins including polyolefins such as medium or high density polyethylene, polypropylene or copolymers thereof, poly-4-methylpentene, and polystyrene. The non-soluble mold component can also be made from polyacetal resins, polyacrylethers, polyarylether sulfones, different types of nylon including nylon 6, nylon 66 and nylon 11. Moreover, the non-soluble mold component can also be made of a corrosion-resistant metallic material such as stainless steel.

In instances where either the first mold component 200 or the second mold component 218 is made of a soluble material and the other mold component is made of a non-soluble material, the method of forming the lens component (e.g., the haptic) can comprise separating the two mold components after the curing step. At least a segment of the soluble core component 236 can be within the molded lens component or partially encapsulated by the molded lens component when the two mold components are separated. For example, the core body 238 of the soluble core component 236 can be within the molded lens component or partially encapsulated by the molded lens component when the molded lens component is removed from the first mold component 200 or the second mold component 218. Moreover, the positioning piece 240 of the soluble core component 236 can be connected to the molded lens component but not contained or encapsulated within the molded lens component.

The method can further comprise immersing the soluble mold component (either the first mold component 200 or the second mold component 218) along with the molded lens component in a solvent to dissolve the soluble core component 236 and the soluble mold component (either the first mold component 200 or the second mold component 218). The method can also comprise heating the solvent to between about 35° C. and 55° C. and agitating the solvent by sonication or stirring to expedite the dissolution of the soluble core component 236 and the soluble mold component. In some embodiments, the soluble core component 236 and the soluble mold component can be dissolved after being immersed in the solvent between about 6 hours to about 24 hours. What remains after all of the soluble material has dissolved is the molded lens component (for example, a haptic having a cavity defined therein).

FIGS. 3A and 3B illustrate perspective views of embodiments of a soluble core component 236. The soluble core component 236 can comprise a curved or arcuate core body 238 connected to a positioning piece 240. In other embodiments not shown in the figures, the core body 238 can be substantially straight or shaped in an undulating or zig-zag manner.

As previously discussed, the positioning piece 240 can elevate or raise the core body 238 such that when the positioning piece 240 is placed on a flat surface, no portion of the core body 238 directly contacts the flat surface.

As shown in FIGS. 3A and 3B, the soluble core component 236 can have a core distal end 242 that is free and not attached to the positioning piece 240. In certain embodiments, the core distal end 242 can be rounded or curved. In other embodiments, the core distal end 242 can be flat, tapered, or pinched. In some embodiments, the soluble core component 236 can comprise two or more positioning pieces 240 and one or more core bodies 238 can extend in between such positioning pieces 240. In these embodiments, the soluble core component 236 does not comprise a free core distal end 242.

As previously discussed, the core body 238 of the soluble core component 236 can be disposed within a component mold cavity formed by the first partial component cavity 204 and the second partial component cavity 222 (see, for example, FIGS. 2A, 2B, and 2D) when the first mold component 200 is mated to the second mold component 218 to form the assembled mold 250. In this manner, the molded lens component (e.g., the haptic) can be formed around the core body 238 of the soluble core component 236.

The positioning piece 240 can be disposed within a positioning cavity formed by the first partial positioning cavity 206 and the second partial positioning cavity 224 (see, for example, FIGS. 2A, 2B, and 2D) when the first mold component 200 is mated to the second mold component 218 to form the assembled mold 250.

As shown in FIGS. 3A and 3B, the positioning piece 240 can comprise a first support wall 300, a second support wall 302, and a bridge component 304 connecting the first support wall 300 to the second support wall 302. The unique shape and design of the positioning piece 240 can allow a manufacturer to more easily remove the soluble core component 236 from a mold component using tweezers or other small plucking or grasping tools. The unique shape and design of the positioning piece 240 can also ensure that the positioning piece 240 does not deform or distort during the injection molding process.

The core body 238 can be connected to an exterior surface of the first support wall 300. The exterior surface of the first support wall 300 can be substantially flat to form a substantially flat optic attachment end to allow the optic attachment end to more easily interface with a substantially flat protruding outer surface of a lens optic portion.

The first support wall 300 of the positioning piece 240 can also act as a barrier to separate the component mold cavity from the positioning cavity within the assembled mold 250. The first support wall 300 can prevent the lens component material within the component mold cavity from coming into contact with lens component material within the positioning cavity. In this manner, the first support wall 300 can serve as a temporary demarcation of the open end of the molded haptic.

The second support wall 302 of the positioning piece 240 can comprise one or more deformable or crushable protuberances 306 extending laterally from an exterior surface of the second support wall 302. The protuberances 306 can create gaps between the exterior surface of the second support wall 302 and the mold component walls defining the positioning cavity. These gaps can ensure the second support wall 302 (and, in turn, the positioning piece 240) does not become stuck or jammed within the positioning cavity. The gaps created by the protuberances 306 can allow a user to more easily remove the positioning piece 240 from either the first partial positioning cavity 206 or the second partial positioning cavity 224 when the first mold component 200 is separated from the second mold component 218.

FIGS. 3A and 3B also illustrate that a bridge component 304 can connect the first support wall 300 to the second support wall 302. The bridge component 304 can connect an inner surface of the first support wall 300 to an inner surface of the second support wall 302. The bridge component 304 can be raised or elevated such that passageways 308 or clearances are formed beneath and above the bridge component 304. In some embodiments, the passageways 308 can allow the low-viscosity lens component material to be injected or otherwise enter the interior of the assembled mold 250 through the passageways 308.

The soluble core component 236 can be made of a soluble or dissolvable material. As previously discussed, at least one of the first mold component 200 and the second mold component 218 can also be made of the same soluble or dissolvable material.

In some embodiments, the soluble core component 236, the first mold component 200, the second mold component 218, or a combination thereof can be made, in whole or in part, of a soluble vinyl alcohol homopolymer. For example, the soluble core component 236, the first mold component 200, the second mold component 218, or a combination thereof can be made, in whole or in part, of a soluble polyvinyl alcohol (PVOH). In some embodiments, the soluble core component 236, the first mold component 200, the second mold component 218, or a combination thereof can be made, in whole or in part, of a soluble polyvinyl alcohol comprising plasticizers. In certain embodiments, the soluble polyvinyl alcohol can comprise glycerol and other additives. More specifically, the soluble core component 236, the first mold component 200, the second mold component 218, or a combination thereof can be made, in whole or in part, of Mowiflex™ C-30 or Mowiflex™ C-600 distributed by Kuraray Europe GmbH.

In other embodiments, the soluble core component 236, the first mold component 200, the second mold component 218, or a combination thereof can be made, in whole or in part, of a butenediol vinyl alcohol (BVOH) co-polymer. The soluble core component 236, the first mold component 200, the second mold component 218, or a combination thereof can also be made, in whole or in part, of a soluble biodegradable material.

The soluble core component 236 can be completely dissolved when immersed in a solvent, such as water, between about 6 hours and 24 hours. The solvent can be heated to between about 35° C. to about 55° C. In other embodiments, the soluble core component 236 can be dissolved in a solvent kept at room temperature. The solvent can also be agitated by sonication to expedite the dissolution of the soluble core component 236. In other embodiments, the solvent can be agitated by stirring the solvent. For example, the soluble core component 236 can be immersed in a heated ultrasonic water bath to dissolve the soluble core component 236. In these and other embodiments, the first mold component 200, the second mold component 218, or a combination thereof can also be dissolved under similar conditions when such mold components are made of a soluble material.

In some embodiments, the entire soluble core component 236 including the positioning piece 240 and the core body 238 can be made of the same soluble material. In other embodiments, the positioning piece 240 or part of the positioning piece 240 can be made of a different soluble material than the core body 238.

Even though the soluble core component 236 is made of a soluble material. The soluble core component 236 can be cured into a substantially hardened or rigid component when used in the molding process. Moreover, when the first mold component 200, the second mold component 218, or a combination thereof are made of the soluble material, the mold component can be cured into a substantially hardened or rigid component when used in the molding process.

FIG. 3B illustrates that the soluble core component 236 can comprise a secondary core feature 310 protruding or extending out from the core body 238. As shown in FIG. 3B, the secondary core feature 310 can be a fin-shaped feature protruding from the radially inner body portion 244 of the core body 238.

The secondary core feature 310 can allow a lens designer to create additional cavities or passageways within a molded lens component. For example, the fin-shaped feature shown in FIG. 3B can be used to create the channel 148 extending into a radially inner portion of the haptic (see, for example, channel 148 of FIGS. 1B and 1C).

FIG. 3B also illustrates that the core body 238 of the soluble core component 236 can comprise a core aperture 312. The core aperture 312 can be a hole or opening along the core body 238 that extends into at least part of the core body 238. As shown in FIG. 3B, the core aperture 312 can be located on the secondary core feature 310.

The core aperture 312 can receive a stabilizing member such as a stabilizing pin 408 (see, for example, FIG. 4) to keep the core body 238 in place during the molding process. For example, a stabilizing pin 408 can be inserted through the core aperture 312 to prevent the core body 238 from shifting or moving during the molding process (for example, when the lens component material is injected into the assembled mold 250 under vacuum).

FIG. 4 illustrates an exploded view of an embodiment of a system 400 for manufacturing a lens component (e.g., the haptic 104). The system 400 can comprise a first mold component 200, a soluble core component 236, a second mold component 218, a third mold component 404, a stabilizing pin 408, a fourth mold component 412, and a clamping member 232. The first mold component 200 can be similar to the first mold component 200 previously described and shown in FIGS. 2A, 2D, and 2E. The soluble core component 236 can be similar to the soluble core component 236 previously described and shown in FIG. 3B.

The second mold component 218 of FIG. 4 can have an elongate opening 402 defined along the second molding surface 220. The elongate opening 402 can be positioned along at least part of the second partial component cavity 222. Part of the second partial component cavity 222 can be missing from the second mold component 218 due to the presence of the elongate opening 402. The elongate opening 402 can be aligned with mold features defined along the third mold component 404.

The second mold component 218 can also have a recessed portion 406 defined along a top side of the second mold component 218. The third mold component 404 can be placed or otherwise positioned within the recessed portion 406 In some embodiments, an initial step in forming an assembled mold using the components of the system 400 can comprise placing or inserting the third mold component 404 into the recessed portion 406 of the second mold component 218. When the third mold component 404 is placed within the recessed portion 406, the third mold component 404 can be detachably coupled to the second mold component 218 via an interference fit.

The soluble core component 236 can then be placed or pressed against the underside of the second mold component 218 comprising the second molding surface 220. For example, the positioning piece 240 of the soluble core component 236 can be pressed into the second partial positioning cavity 224 (not shown in FIG. 4) defined along the second molding surface 220 and the core body 238 of the soluble core component 236 can be positioned partly within the second partial component cavity 224 but not in physical with the second molding surface 220.

The soluble core component 236 can be detachably coupled to the second mold component 218 via an interference fit when the positioning piece 240 is pressed or otherwise inserted into the second partial positioning cavity 224.

With the soluble core component 236 detachably coupled to the second mold component 218, a stabilizing pin 408 can be inserted through a core aperture 312 defined along the core body 238 or a secondary core feature 310 extending or protruding from the core body 238. The stabilizing pin 408 can be inserted through the core aperture 312 to prevent the core body 238 from shifting or moving during the molding process. For example, the stabilizing pin 408 can be made in part of a metallic material. The stabilizing pin 408 can have a diameter between about 0.5 mm to about 2.0 mm. The stabilizing pin 408 can be inserted into a receiving conduit or space defined along an underside of the third mold component 404.

The third mold component 404 can comprise a third molding surface (not shown in FIG. 4) with surface features, cavities, or a combination thereof defined along the third molding surface. For example, the third mold component 404 can be used to introduce negative space into parts of the molded lens component.

The second mold component 218 comprising the third mold component 404 and the soluble core component 236 can then be mated with the first mold component 200 to form a first assembled mold. The clamping member 232 can then be detachably fastened to the first mold component 200 to form an enclosed chamber. As shown in FIG. 4, the clamping member 232 (for example, a clamping cap) can have a septum 410 or opening defined along a top of the clamping member 232 to allow an injector nozzle to enter through the septum 410 or opening to deliver a first curable material, such as the lens component material previously discussed (e.g., the haptic material), into the enclosed chamber. For example, the first curable material can be injected or introduced into the reservoir space 214 of the first mold component 200.

The enclosed chamber comprising the clamping member 232 coupled to the first assembled mold can be cured to form a preliminary molded lens component. The clamping member 232 and the third mold component 404 can then be separated from the first mold component 200 and the second mold component 218. At this point, a fourth mold component 412 comprising a fourth molding surface can be placed or otherwise positioned within the recessed portion 406 of the second mold component 218. The fourth mold surface can comprise surface features and cavities that can be used to define the shape or positioning of additional portions of the molded lens component. The additional portions of the molded lens component can be made of a second curable material. The second curable material can be different than the first curable material (or the lens component material) used to make the preliminary molded lens component. In some embodiments, the second curable material can comprise or be the composite material disclosed herein.

In some embodiments, the fourth mold component 412 can comprise openings or ports defined along the surface of the fourth mold component 412 to allow an injector nozzle or fluid delivery device to deliver the second curable material into cavities created by the fourth molding surface and the negative space defined along the preliminary molded lens. The same clamping member 232 or another clamping member 232 can then be detachably coupled to the first mold component 200 to form a second assembled mold. The second assembled mold can then be cured to form the molded lens component. This molded lens component comprising the soluble core component 236 or a combination of the molded lens component comprising the soluble core component 236 and any or all of the first mold component 200, the second mold component 218, and the fourth mold component 412 (depending on whether any of the mold components are made of the soluble material) can be immersed in the solvent to dissolve the soluble core component 236 or one or more of the mold components.

Although the system 400 shown in FIG. 4 includes four mold components (e.g., the first mold component 200, the second mold component 218, the third mold component 404, and the fourth mold component 412), it is contemplated by this disclosure that the system 400 can comprise more mold components including a fifth mold component, a sixth mold component, a seventh mold component, an eighth mold component, or nine or more mold components. These additional mold components can be used to form even more complex or elaborate molded lens components comprising different portions made of different curable materials, a plurality of interior chambers, or a combination thereof.

For example, a variation of the system 400 shown in FIG. 4 can be used to form the haptic(s) 104 shown in FIGS. 1A-1C.

One technical problem faced by the applicants is how to form haptics comprising complex internal shapes and geometries and differing anterior and posterior textures. One technical solution discovered and developed by the applicants is to form the haptics using the soluble cores and mold components disclosed herein. Moreover, the haptics formed using the soluble cores and mold components disclosed herein can be coupled to an optic portion comprising an optic fluid chamber to form an IOL (e.g., an accommodating IOL) that is optimized for placement within a subject's capsular bag and that is adapted to deform in response to ciliary muscle movement. A haptic formed using the soluble cores and molds disclosed herein are well suited for such deformations.

FIG. 5A is an image showing a top-down view of an anterior portion of one embodiment of a haptic. The anterior portion of the haptic can also be referred to herein as the haptic anterior portion 119. In the embodiment shown in FIG. 5A, at least part of the haptic anterior portion 119 can be characterized by a smooth or polished texture or finish.

The surface profile of the haptic anterior portion 119 can be measured using a variety of techniques including, but not limited to, light interferometry and high-resolution microscopy.

In some embodiments, the surface roughness of the haptic anterior portion 119 can be quantified in Ra (roughness average) units in micrometers (μm) and graded using a Society of the Plastics Industry (SPI) grade value.

In certain embodiments, the surface roughness of the haptic anterior portion 119, as measured in Ra units, can be between about 0.10 μm and about 0.35 μm. More specifically, the surface roughness of the haptic anterior portion 119, as measured in Ra units, can be between about 0.14 μm and about 0.35 μm.

In these and other embodiments, the surface roughness of the haptic anterior portion 119 can be graded at an SPI grade of between B1 and C1 (e.g., any of B1, B2, B3, or C1). More specifically, the surface roughness of the haptic anterior portion 119 can be graded at an SPI grade of between B1 and C1.

FIG. 5B is an image showing a top-down view of a posterior portion of the haptic shown in FIG. 5A. The posterior portion of the haptic can also be referred to herein as the haptic posterior portion 121. In the embodiment shown in FIG. 5B, at least part of the haptic posterior portion 121 can be characterized by a frosted or coarse texture or finish.

The surface profile of the haptic posterior portion 121 can also be measured using a variety of techniques including, but not limited to, light interferometry and high-resolution microscopy.

In some embodiments, the surface roughness of the haptic posterior portion 121 can be quantified in Ra units in μm and graded using a SPI grade value. The surface roughness of the haptic posterior portion 121 can be greater than the surface roughness of the haptic anterior portion 119 such that at least part of the haptic anterior portion 119 is smoother or more polished than the haptic posterior portion 121.

In certain embodiments, the surface roughness of the haptic posterior portion 121, as measured in Ra units, can be between about 0.25 μm and about 0.60 μm. More specifically, the surface roughness of the haptic posterior portion 121, as measured in Ra units, can be between about 0.27 μm and about 0.57 μm.

In these and other embodiments, the surface roughness of the haptic posterior portion 121 can be graded at an SPI grade of between B3 and C3 (e.g., any of B3, C1, C2, or C3). More specifically, the surface roughness of the haptic posterior portion 121 can be graded at an SPI grade of between B3 and C2.

FIGS. 5A and 5B also illustrate that part of the haptic 104 can be made of the composite material (shown as the slightly darker region along the radially inner portion of the haptic 104). Also shown in FIGS. 5A and 5B are the haptic isolators 105.

FIG. 6 is an image showing a close-up side view of part of the haptic 104. As shown in FIG. 6, the haptic 104 can comprise a haptic anterior portion 119 and a haptic posterior portion 121. The haptic anterior portion 119 can be substantially transparent or more transparent than the haptic posterior portion 121. The haptic posterior portion 121 can be substantially translucent.

In some embodiments, the haptic anterior portion 119 and the haptic posterior portion 121 can both be considered translucent but the haptic anterior portion 119 can allow more laser light to penetrate through the haptic anterior portion 119 without interfering with the laser light (as opposed to the haptic posterior portion 121).

In some embodiments, a thickness or depth of the haptic anterior portion 119 can be between about 600 μm and about 850 μm (e.g., about 830 μm).

In some embodiments, a thickness or depth of the haptic posterior portion 121 can be between about 600 μm and about 850 μm (e.g., about 830 μm).

As shown in FIG. 6, at least part of the haptic 104 can be made of the composite material disclosed herein. As previously discussed, the composite material can be configured to change shape in response to receiving laser light (e.g., laser light 125, see FIGS. 1B and 1C) directed at the composite material and a base power of the optic portion 102 of the IOL 100 can be configured to change in response to this shape change caused by the laser light 125 directed at the composite material.

In some embodiments, the composite material can be positioned anterior to the haptic posterior portion 121. In these and other embodiments, the composite material can be positioned posterior to the haptic anterior portion 119.

One technical advantage provided by the frosted or translucent haptic posterior portion 121 is that the haptic posterior portion 121 can act as a safety feature to diffuse errant laser energy from any missed laser shots directed at the composite material. This may reduce the likelihood of inadvertently damaging the retina of the patient. The diffuser effect of the frosted surface of the haptic posterior portion 121 can be quantified using radiometry (to measure light intensity) and UV-vis spectrophotometry (to measure light transmittance or reflectance). In some cases, a beam profiler can also be used to analyze the shape of the laser beam after it passes through the frosted or translucent haptic posterior portion 121 and compare this beam shape against the same laser beam that passes through the smooth haptic anterior portion 119.

Another technical advantage discovered by the applicant is that the polished haptic anterior portion 119 and the frosted haptic posterior portion 121 can work together to enhance the visualization of the composite material and the various features within the haptic 104. The haptic posterior portion 121 can serve as a frosted or diffuse background which allows a clinician to more easily see the composite material and the various features within the haptic 104 during a clinical procedure.

FIG. 7A is an image showing part of the first mold component 200 and FIG. 7B is an image showing a close-up view of part of the first molding surface 202 of the first mold component 200.

At least part of the first molding surface 202, including the first partial component cavity 204, can have a first surface roughness. In some embodiments, the first partial component cavity 204 can have the same first surface roughness as the remainder of the first molding surface 202.

In some embodiments, the surface profile the first molding surface 202, including the first partial component cavity 204, can be measured using techniques such as light interferometry and high-resolution microscopy.

In some embodiments, the surface roughness of the first molding surface 202, including the first partial component cavity 204, can be quantified in Ra units in μm and graded using a SPI grade value.

In certain embodiments, the first surface roughness, as measured in Ra units, can be between about 0.10 μm and about 0.35 μm. More specifically, the first surface roughness, as measured in Ra units, can be between about 0.10 μm and about 0.32 μm.

In these and other embodiments, the first surface roughness can be graded at an SPI grade of between B1 and C1 (e.g., any of B1, B2, B3, or C1). More specifically, the first surface roughness can be graded at an SPI grade of B2 or B3.

The first molding surface 202, including the first partial component cavity 204, can impart a smooth or polished texture or finish to the haptic anterior portion 119 once the haptic 104 is removed from the assembled mold after the curing step.

The smooth or polished texture of the first molding surface 202, including the first partial component cavity 204, can be imparted by a steel mold used to make the first mold component 200. Thus, the surface roughness or surface texture of at least part of the steel mold used to make the first mold component 200 would also need to be controlled or optimized to impart a smooth or polished texture to the first molding surface 202 of the first mold component 200.

FIG. 8A is an image showing part of the second mold component 218 and FIG. 8B is an image showing a close-up view of part of the second molding surface 220 of the second mold component 218. At least part of the second molding surface 220, including the second partial component cavity 222, can have a second surface roughness. In some embodiments, the second partial component cavity 222 can have the same second surface roughness as the remainder of the second molding surface 220.

In some embodiments, the surface profile of the second molding surface 220, including the second partial component cavity 222, can be measured using techniques such as light interferometry and high-resolution microscopy.

In some embodiments, the surface roughness of the second molding surface 220, including the second partial component cavity 222, can be quantified in Ra units in μm and graded using a SPI grade value.

In certain embodiments, the second surface roughness, as measured in Ra units, can be between about 0.25 μm and about 0.60 μm. More specifically, the second surface roughness, as measured in Ra units, can be between about 0.35 μm and about 0.55 μm.

In these and other embodiments, the second surface roughness can be graded at an SPI grade of between B3 and C3 (e.g., any of B3, C1, C2, or C3). More specifically, the second surface roughness can be graded at an SPI grade of C1 or C2.

The second surface roughness can be greater than the first surface roughness such that at least part of the first molding surface 202 can be smoother than the second molding surface 220 and the second molding surface 220 can be rougher or coarser than the first molding surface 202.

The second molding surface 220, including the second partial component cavity 222, can impart a frosted or coarse texture or finish to the haptic posterior portion 121 once the haptic 104 is removed from the assembled mold after the curing step.

The frosted or coarse texture or finish of the second molding surface 220, including the second partial component cavity 222, can be imparted by a steel mold used to make the second mold component 218. Thus, the surface roughness or surface texture of at least part of the steel mold used to make the second mold component 218 would also need to be controlled or optimized to impart a rough or coarse texture to the second molding surface 220 of the second mold component 218.

At a high-level, the first mold component 200 and the second mold component 218 (also referred to as the first and second wafers or A and B wafers) can be plastic mold halves that are configured to be mated or assembled together with a soluble core (e.g., the soluble core component 236) in the middle. A liquid formulation of the haptic material disclosed herein can be dispensed into the reservoir formed by the assembled mold and cured. After curing, the mold halves (the first mold component 200 and the second mold component 218) can be de-molded and the soluble core (e.g., the soluble core component 236) can be dissolved away to yield the haptic 104.

A number of embodiments have been described. Nevertheless, it will be understood by one of ordinary skill in the art that various changes and modifications can be made to this disclosure without departing from the spirit and scope of the embodiments. Elements of systems, devices, apparatus, and methods shown with any embodiment are exemplary for the specific embodiment and can be used in combination or otherwise on other embodiments within this disclosure. For example, the steps of any methods depicted in the figures or described in this disclosure do not require the particular order or sequential order shown or described to achieve the desired results. In addition, other steps or operations may be provided, or steps or operations may be eliminated or omitted from the described methods or processes to achieve the desired results. Moreover, any components or parts of any apparatus or systems described in this disclosure or depicted in the figures may be removed, eliminated, or omitted to achieve the desired results. In addition, certain components or parts of the systems, devices, or apparatus shown or described herein have been omitted for the sake of succinctness and clarity.

Accordingly, other embodiments are within the scope of the following claims and the specification and/or drawings may be regarded in an illustrative rather than a restrictive sense.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit, or scope of the present invention.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the invention. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments therebetween.

All existing subject matter mentioned herein (e.g., publications, patents, patent applications) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “an,” “said,” and “the” include plural referents unless the context clearly dictates otherwise. It is further noted that the claims may be drafted to exclude any optional element.

As such, this statement is intended to serve as antecedent basis for use of such exclusive terminology as “solely,” “only,” and the like in connection with the recitation of claim elements, or use of a “negative” limitation. Unless defined otherwise, all technical and scientific terms used herein have the same meaning as commonly understood by one of ordinary skill in the art to which this invention belongs.

Reference to the phrase “at least one of” when such phrase modifies a plurality of items or components (or an enumerated list of items or components) means any combination of one or more of those items or components. For example, the phrase “at least one of A, B, and C” means: (i) A; (ii) B; (iii) C; (iv) A, B, and C; (v) A and B; (vi) B and C; or (vii) A and C.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including,” “having,” and their derivatives. Also, the terms “part,” “section,” “portion,” “member,” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved.

Finally, terms of degree such as “substantially,” “about,” and “approximately” as used herein mean the specified value or the specified value and a reasonable amount of deviation from the specified value (e.g., a deviation of up to ±0.1%, ±1%, ±5%, or ±10%, as such variations are appropriate) such that the end result is not significantly or materially changed. For example, “about 1.0 cm” can be interpreted to mean “1.0 cm” or between “0.9 cm and 1.1 cm.” When terms of degree such as “about” or “approximately” are used to refer to numbers or values that are part of a range, the term can be used to modify both the minimum and maximum numbers or values.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

1. An intraocular lens, comprising:
an optic portion; and
a haptic coupled to the optic portion,
wherein the haptic comprises a haptic anterior portion and a haptic posterior portion,
wherein the haptic anterior portion has an anterior surface roughness and the haptic posterior portion has a posterior surface roughness, wherein the posterior surface roughness is greater than the anterior surface roughness such that at least part of the haptic anterior portion is smoother than the haptic posterior portion.

2. The intraocular lens of claim 1, wherein the anterior surface roughness has a roughness average (Ra) of between about 0.10 μm and about 0.35 μm.

3. The intraocular lens of claim 1, wherein the anterior surface roughness is graded at a Society of the Plastics Industry (SPI) grade of between B1 and C1.

4. The intraocular lens of claim 1, wherein the posterior surface roughness has a roughness average (Ra) of between about 0.25 μm and about 0.60 μm.

5. The intraocular lens of claim 1, wherein the posterior surface roughness is graded at a Society of the Plastics Industry (SPI) grade of between B3 and C3.

6. The intraocular lens of claim 1, wherein a thickness or depth of the haptic anterior portion is between about 600 μm and about 850 μm.

7. The intraocular lens of claim 1, wherein a thickness or depth of the haptic posterior portion is between about 600 μm and about 850 μm.

8. The intraocular lens of claim 1, wherein the optic portion comprises an optic fluid chamber, and wherein the haptic comprises a haptic lumen extending through at least part of the haptic and in fluid communication with the optic fluid chamber.

9. The intraocular lens of claim 1, wherein at least part of the haptic is made of a composite material.

10. The intraocular lens of claim 9, wherein the composite material is positioned anterior to the haptic posterior portion.

11. The intraocular lens of claim 9, wherein the composite material is positioned posterior to the haptic anterior portion.

12. The intraocular lens of claim 9, wherein the composite material is configured to change shape in response to receiving laser light directed at the composite material, and wherein a base power of the optic portion is configured to change in response to the laser light directed at the composite material.

13. The intraocular lens of claim 12, wherein the haptic posterior portion is configured to diffuse the laser light directed at the haptic.

14. The intraocular lens of claim 1, wherein at least part of the haptic anterior portion is characterized by a smooth texture or finish.

15. The intraocular lens of claim 14, wherein the smooth texture or finish is imparted by a first molding surface of a first mold component.

16. The intraocular lens of claim 15, wherein at least part of the haptic posterior portion is characterized by a frosted texture or finish.

17. The intraocular lens of claim 16, wherein the frosted texture or finish is imparted by a second molding surface of a second mold component configured to couple to or mate with the first mold component.

18. (canceled)

19. (canceled)

20. A system for manufacturing a lens component, comprising:
a first mold component comprising a first molding surface defined by a first partial component cavity, wherein at least part of the first molding surface has a first surface roughness; and
a second mold component comprising a second molding surface defined by a second partial component cavity, wherein at least part of the second molding surface has a second surface roughness,
wherein the second surface roughness is greater than the first surface roughness such that at least part of the first molding surface is smoother than the second molding surface,
wherein the first mold component is configured to be mated to the second mold component to form an assembled mold, and
wherein a mold cavity within the assembled mold is configured to receive a lens component material for curing into a molded lens component.

21.-30. (canceled)

31. A method of manufacturing a lens component, comprising:
providing a first mold component comprising a first molding surface defined by a first partial component cavity, wherein at least part of the first molding surface has a first surface roughness;
providing a second mold component comprising a second molding surface defined by a second partial component cavity, wherein at least part of the second molding surface has a second surface roughness, and wherein the second surface roughness is greater than the first surface roughness such that the first molding surface is smoother than the second molding surface;
mating a second mold component to the first mold component to form an assembled mold;
introducing a lens component material into a mold cavity; and
curing the lens component material within the assembled mold to form a molded lens component.

32.-40. (canceled)

41. An intraocular lens haptic, comprising:
a haptic anterior portion; and
a haptic posterior portion,
wherein the haptic anterior portion has an anterior surface roughness and the haptic posterior portion has a posterior surface roughness, wherein the posterior surface roughness is greater than the anterior surface roughness such that at least part of the haptic anterior portion is smoother than the haptic posterior portion.

42.-58. (canceled)
